(19) United States
(12) Patent Application Publication
(10) Pub. No.: US 2014/0303036 A1
(43) Pub. Date: Oct. 9, 2014

(54) Nucleic Acid Assembly System

(71) Applicant: DSM IP ASSETS B.V., Heerlen (NL)

(72) Inventors: Johannes Andries Roubos, Echt (NL); Bernard Meijrink, Echt (NL); Richard Kerkman, Echt (NL); Ben Den Dulk, Echt (NL)

(21) Appl. No.: 14/359,358

(22) PCT Filed: Nov. 23, 2012
(86) PCT No.: PCT/EP2012/073532

§ 371 Date: May 20, 2014

Related U.S. Application Data
(60) Provisional application No. 61/563,146, filed on Nov. 23, 2011.

(30) Foreign Application Priority Data
Nov. 23, 2011 (EP) 11190372.0

(51) Int. Cl. C12N 15/10

(52) U.S. Cl. CPC C12N 15/1082
USPC 506/10; 506/26; 506/14; 506/17; 536/23.1; 435/254.2

(57) ABSTRACT

The present invention relates to a method for the preparation of a library of host cells, a plurality of which comprise an assembled polynucleotide at a target locus, which method comprises: (a) providing a plurality of polynucleotides comprising two or more polynucleotide subgroups, wherein: (i) a plurality of polynucleotides in each polynucleotide subgroup comprises sequence encoding a peptide or polypeptide and/or a regulatory sequence; (ii) a plurality of peptides or polypeptides encoded by, or a plurality of regulatory sequences comprised within, each polynucleotide subgroup share an activity and/or function; (iii) at least one polynucleotide subgroup comprises at least two non-identical polynucleotide species; (iv) a plurality of polynucleotides of each polynucleotide subgroup comprises sequence enabling homologous recombination with a plurality of polynucleotides from one or more other polynucleotide subgroups; and (v) a plurality of polynucleotides in two polynucleotide subgroups comprise a nucleotide sequence enabling homologous recombination with a target locus in host cells; and (b) assembling the plurality of polynucleotides at the target locus by homologous recombination in vivo in host cells, thereby to generate a library of host cells, a plurality of which comprise an assembled polynucleotide at the target locus. The assembled polynucleotides may be recovered, thereby to prepare a library of nucleic acids.

### FIELD OF THE INVENTION

The present invention relates to a method for the preparation of a library of host cells. The invention also relates to a method for the preparation of a library of nucleic acids and to a method for the preparation of a host cell having a desired property. The invention further relates to a library of host cells, a library of nucleic acids and a host cell having a desired property prepared according to such methods.

### BACKGROUND OF THE INVENTION

Organisms, and in particular, microorganisms, may be used to produce biological and chemical products, sometimes with less expense and with less environmental impact than using chemical synthesis or petroleum based chemistries. Some microorganisms offer an advantage of being amenable to genetic modification. Microorganisms can be engineered to produce products of interest by harnessing native or modified metabolic pathways, and by introducing novel pathways.

In a given pathway, multiple polypeptides have activities that convert a substrate to a product via a series of intermediates. Many microorganisms have similar, if not identical pathways, yet a particular type of activity at a parallel step in a pathway may be carried out with more or less efficiency when comparing two different organisms. For two organisms sharing a common pathway, for example, counterpart polypeptides that that are responsible for a parallel activity in the pathway may affect the activity with a different efficiency or different rate. Thus, while related or unrelated organisms may have similar or identical pathways, the efficiency or rate at which each activity is affected may differ among microorganisms.

Methods are required in which this natural variation and other types of variation may be exploited.

### SUMMARY OF THE INVENTION

Provided herein are methods useful for optimizing one or more pathways in an engineered microorganism. In particular, the methods may be utilized to optimize production of a target product by an engineered microorganism. For two or more activities or functions in a pathway, the methods herein provide different combinations of polypeptides (and regulatory sequences controlling expression of those polypeptides) that carry out the activities/functions in an organism.

Of these, combinations that give rise to efficient production of target product can be identified and selected, thereby producing organisms with optimized production of the target product.

Critically, the combinations are assembled in host cells in vivo, such that the methods of the invention provide a quick, efficient strategy for generating genetic diversity which may readily be screened for a desired property. The invention thus provides a method in which a library of host cells may be screened for a desired property. Such a method may comprise determining the amount of a target product produced by the host cells in the library.

In the invention, a number of polynucleotide subgroups are provided. The polynucleotide subgroups are such that each polynucleotide in a subgroup is capable of homologous recombination with polynucleotides from one or more other groups. In addition, the polynucleotides from two groups are capable of homologous recombination with a target site in the host cells. Accordingly, the method of the invention allows assembled polynucleotides to be generated which typically each comprise a polynucleotide from each of the subgroups and which are incorporated by homologous recombination at a target locus within a host cell.

Variation can be introduced into one or more polynucleotide subgroups. That is to say a polynucleotide subgroup may comprise two or more non-identical sequences. Thus, by allowing the polynucleotide subgroups to undergo homologous recombination, variant assembled polynucleotides may be generated. The polynucleotide subgroups are assembled in vivo such that a library of host cells is generated comprising variant assembled polynucleotides.

The host cells may be screened to identify a host cell with a desired property conferred by the assembled polynucleotide comprised within that host cell. For example, an assembled polynucleotide may comprise sequences encoding the various members of a pathway. The method can thus be used to identify variant combinations of the members of the pathway that are give rise to, for example, efficient production of a target product.

According to the invention, there is thus provided a method for the preparation of a library of host cells, a plurality of which comprise an assembled polynucleotide at a target locus, which method comprises:

(a) providing a plurality of polynucleotides comprising two or more polynucleotide subgroups, wherein:

(i) a plurality of polynucleotides in each polynucleotide subgroup comprises sequence encoding a peptide or polypeptide and/or a regulatory sequence;

(ii) a plurality of peptides or polypeptides encoded by, or a plurality of regulatory sequences comprised within, each polynucleotide subgroup share an activity and/or function;

(iii) at least one polynucleotide subgroup comprises at least two non-identical polynucleotide species;

(iv) a plurality of polynucleotides of each polynucleotide subgroup comprises sequence enabling homologous recombination with a plurality of polynucleotides from one or more other polynucleotide subgroups; and

(v) a plurality of polynucleotides in two polynucleotide subgroups comprise a nucleotide sequence enabling homologous recombination with a target locus in host cells; and

(b) assembling the plurality of polynucleotides at the target locus by homologous recombination in vivo in host cells,

thereby to generate a library of host cells, a plurality of which comprise an assembled polynucleotide at the target locus.

The invention also provides:

a method for the preparation of a library of assembled polynucleotides, which method comprises:
preparing a library of host cells according to the method of the invention; and
recovering the assembled polynucleotides from the library of host cells, thereby to prepare a library of assembled polynucleotides;

a method for the preparation of a host cell having a desired property, which method comprises:
preparing a library of host cells according to the method of the invention; and
screening said library of host cells, thereby to identify a host cell with the desired property;

a method for the preparation of a host cell having a desired property, which method comprises:
preparing a library of assembled polynucleotides according to the method of the invention;
transferring the library into host cells; and
screening the resulting host cells, thereby to identify a host cell with the desired property; and

a method for expression screening of filamentous fungal transformants, comprising:
(a) isolating single colony transformants of a library of yeast host cells prepared by a method according to the invention;
(b) preparing DNA from the single colony of yeast transformants;
(c) introducing a sample of the preparations of step (b) into separate suspensions of protoplasts of a filamentous fungus to obtain transformants thereof, wherein transformants contain one or more copies of an individual polynucleotide from the library of yeast host cells;
(d) growing the individual filamentous fungal transformants of step (c) on selective growth medium, thereby permitting growth of the filamentous fungal transformants, while suppressing growth of untransformed filamentous fungi; and
(e) measuring activity or a property of each polypeptide encoded by the individual polynucleotides

Also, the invention relates to a library of host cells, a library of nucleic acids and a host cell having a desired property prepared according to the methods of the invention.

### BRIEF DESCRIPTION OF THE FIGURES

FIG. 1 shows an example for the assembly of variant nucleic acids, adding variations into a pathway by adding multiple fragments as option for recombining a pathway and integrating the selectable marker (in this case KanMX), afterwards screen all strains obtained from transformation and find the best combinations and or learn from all obtained results to improve a final pathway.

FIG. 2 shows the test pathway. HIS3 functions as a selective marker after transformation, all other parts in the pathway are easy to score on phenotype and can be used therefore to demonstrate the principle of adding variation into a pathway.

FIG. 3 shows the cassettes of Example X that can integrate via homologous recombination into the yeast genome. The light grey on the edge of each cassette depicts 50-bp homology regions that are applied for in vivo homologous recombination.

FIG. 4 shows PCR reactions of PCR reaction 1 and 2 analyzed on gel. The numbers at each lane refer to the numbers in Table 2.

FIG. 5 shows PCR reactions of PCR reaction 2 analyzed on gel. The numbers at each lane refer to the numbers in Table 2.

FIG. 6 shows PCR reactions of PCR reaction 3 and the EcoRV cut of PCR reaction 3 analyzed on gel. The numbers at each lane refer to the numbers in Table 2.

FIG. 7 shows PCR reaction 3 cut with EcoRV analyzed on gel. The numbers at each lane refer to the numbers in Table 2.

### DESCRIPTION OF THE SEQUENCE LISTING

SEQ ID NO: 1 to SEQ ID NO: 14 are described in Table 1.

SEQ ID NO: 15 PCR sets out the nucleic acid sequence of the fragment “5′ ADE1 flank” with homology to part 1 (HIS3) in the test pathway.

SEQ ID NO: 16 sets out nucleic acid sequence of the PCR fragment “3′ ADE1 flank” with homology to part 5 (URA3) in the test pathway.

SEQ ID NO: 17 sets out the nucleic acid sequence of the HIS3 expression cassette

SEQ ID NO: 18 sets out the nucleic acid sequence of the LEU2 expression cassette.

SEQ ID NO: 19 sets out the nucleic acid sequence of the Kanmx expression cassette (G418 resistance).

SEQ ID NO: 20 sets out the nucleic acid sequence of the ble expression cassette (phleomycin resistance).

SEQ ID NO: 21 sets out the nucleic acid sequence of the Nat1 expression cassette (Nourseothricin resistance).

SEQ ID NO: 22 sets out the nucleic acid sequence of the Hygromycin resistance expression cassette.

SEQ ID NO: 23 sets out the nucleic acid sequence of the TRP1 expression cassette.

SEQ ID NO: 24 sets out the nucleic acid sequence of the URA3 expression cassette.

SEQ ID NOs: 25 to 42 set out the sequences of the primers used to amplify the designed cassettes and the integration flanks in Example 2.

SEQ ID NOs: 43 to 54 set out the sequences of the expression cassettes (promoter, open reading frame and terminator) used to form the pathway variants described in Example 2.

SEQ ID NOs: 55 and 56 set out the primers in the PCR reactions used to determine the presence of cassette 120 or cassette 121 in Example 2.

SEQ ID NOs: 57 to 63 set out the primers in the PCR reactions used to determine the presence of various cassettes in Example 2.

### DETAILED DESCRIPTION OF THE INVENTION

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

Provided by the invention are methods for the generation of libraries of host cells and nucleic acids, particularly assembled polynucleotides.

Such libraries may be used to identify microorganisms which, for example, are optimized for the production of a desired target product. That is to say, the invention provides methods for optimizing or improving one or more pathways in an engineered microorganism, and can be utilized to optimize or improve production of a target product by an engineered microorganism.

For activities/functions in a pathway, methods herein provide different combinations of polypeptide encoding polynucleotides (that carry out those activities/functions in an organism) and/or combinations of the regulatory sequences that control expression of the polypeptides encoded by such polynucleotides. Of these, combinations that give rise to efficient production of target product may be identified and selected, thereby providing organisms with optimized production of a desired target product.

The methods described herein provide multiple combinations of possible pathways by providing variation for at least one position within a pathway. These methods may be referred to as “combinatorial methods.” Thus, the methods described herein can be used to improve or optimize target product formation in an engineered organism. The terms “improve” and “optimization,” and similar terms, as used herein, refer to a method in which whereby a metabolic pathway or portion thereof, is altered using naturally occurring and/or synthesized polynucleotides (e.g., engineered genetic diversity) to increase the rate, yield, and/or production efficiency of a desired end product, when compared to native or reference activities.

The method of the invention, for such improvement or optimization, is described in further detail herein. In particular, subgroups of polynucleotides are generated, one or more of which may comprise variation. Combinations of polynucleotides from the subgroups may be generated, the combinations assembled in vivo and expressed in host cells. The resulting host cells may then be tested to determine which of the combinations more efficiently or effectively produce a target product.

The term “pathway”, as used herein, is to be interpreted broadly, and may refer to a series of simultaneous, sequential or separate chemical reactions, effected by activities that convert substrates or beginning elements into end compounds or desired products via one or more intermediates. An activity sometimes is conversion of a substrate to an intermediate or product (e.g., catalytic conversion by an enzyme) and sometimes is binding of molecule or ligand, in certain embodiments. The term “identical pathway” as used herein, refers to pathways from related or unrelated organisms that have the same number and type of activities and result in the same end product. The term “similar pathway” as used herein, refers to pathways from related or unrelated organisms that have one or more of: a different number of activities, different types of activities, utilize the same starting or intermediate molecules, and/or result in the same end product.

Pathway improvement and optimization can be attained, for example, by harnessing naturally occurring genetic diversity and/or engineered genetic diversity. Naturally occurring genetic diversity can be harnessed by testing subgroup polynucleotides from different organisms. Engineered genetic diversity can be harnessed by testing subgroup polynucleotides that have been codon-optimized or mutated, for example. For codon-optimized diversity, amino acid codon triplets can be substituted for other codons, and/or certain nucleotide sequences can be added, removed or substituted. For example, native codons may be substituted for more or less preferred codons. In certain embodiments, pathways can be optimized by substituting a related or similar activity for one or more steps from a similar but not identical pathway. A polynucleotide in a subgroup also may have been genetically altered such that, when encoded, effects an activity different than the activity of a native counterpart that was utilized as a starting material for genetic alteration. Nucleic acid and/or amino acid sequences altered by the hand of a person as known in the art can be referred to as “engineered” genetic diversity.

A metabolic pathway can be seen as a series of reaction steps which convert a beginning substrate or element into a final product. Each step may be catalyzed by one or more activities. In a pathway where substrate A is converted to end product D, intermediates B and C are produced and converted by specific activities in the pathway. Each specific activity of a pathway can be considered a species of an activity subgroup and a polypeptide that encodes the activity can be considered a species of a counterpart polypeptide subgroup.

Any peptides, polypeptides or proteins, or an activity catalyzed by one or more peptides, polypeptides or proteins may be encoded by a polynucleotide subgroup. Representative proteins include enzymes (e.g., part or all of a metabolic pathway), antibodies, serum proteins (e.g., albumin), membrane bound proteins, hormones (e.g., growth hormone, erythropoietin, insulin, etc.), cytokines, etc., and include both naturally occurring and exogenously expressed polypeptides. Representative activities (e.g., enzymes or combinations of enzymes which are functionally associated to provide an activity or group of activities as in a metabolic pathway) include any activities associated with a desired metabolic pathway. The term “enzyme” as used herein may refer to a protein which can act as a catalyst to induce a chemical change in other compounds, thereby producing one or more products from one or more substrates.

It will be understood that the methods and compositions described in embodiments presented herein can be used to; (i) optimize any metabolic pathway that produces a desirable end product, and/or (ii) optimize subdomains within an activity subgroup of a metabolic pathway. The term “protein” as used herein refers to a molecule having a sequence of amino acids linked by peptide bonds. This term includes fusion proteins, oligopeptides, peptides, cyclic peptides, polypeptides and polypeptide derivatives, whether native or recombinant, and also includes fragments, derivatives, homologs, and variants thereof. A protein or polypeptide sometimes is of intracellular origin (e.g., located in the nucleus, cytosol, or interstitial space of host cells in vivo) and sometimes is a cell membrane protein in vivo. In some embodiments (described above, and in further detail below in Engineering and Alteration Methods), a genetic modification can result in a modification (e.g., increase, substantially increase, decrease or substantially decrease) of a target activity.

As organisms evolve, in different environments and with different selective pressures, the nucleic acid and amino acid sequences of organisms also can evolve and diverge from an ancestral type. Sequence evolution can result in metabolic pathways that may be naturally optimized for a particular organism in a particular environment, which contributes to the genetic diversity of the respective pathways. Changes in nucleotide or amino acid sequences sometimes may cause the efficiency of an activity to be altered (e.g., increase or decrease in the number of number of conversions or energy input/output of the reaction, for example). The changes may have occurred as a result of different selective pressures with which divergently evolving organisms were presented. These selective pressures may have selected for altered activity that allowed the organism containing the altered sequences to function better in a particular environment. These changes increase genetic diversity of similar or identical activities. The evolutionary changes of similar or identical activities can be identified by nucleic acid and/or amino acid sequence comparisons of related activities from organisms with similar or identical pathways. This evolutionary-driven genetic diversity is referred to herein as “natural diversity.” Commercially useful organisms may have differences in cellular machinery when compared to organisms from which donor activities can be obtained (e.g., transcription and/or translation machinery, for example). An optimized metabolic pathway can be generated for a chosen host organism by combining similar or identical activities from different sources (e.g., natural or engineered genetic diversity), and identifying those combinations that show improvements according to a chosen criteria (e.g., changes in the rate of reaction, changes in yield of reaction, changes in energy requirements for a reaction or efficiency of reaction, and the like or combinations thereof, for example).

In addition to metabolic pathway optimization, the method of the invention may also be used to optimize individual subgroup activities. Thus, each subgroup activity, represented by a polypeptide, can be further divided into further subgroups. The polypeptide domains can represent all or a portion of known activity centers, contact residues and the like.

Oligonucleotides encoding codon optimized versions of the amino acids in each subdomain from each organism also can be synthesized and assembled in various combinations to further optimize individual activity subgroups. For example, conventional recombinant DNA methods (e.g., cloning, PCR, library construction and the like, for example) can be used to generate the polypeptide subdomain libraries for each activity subgroup. By using recombinant DNA techniques available to one of skill in the art, or oligos of a particular target length and configuration to allow self assembly, various regions of each activity may be further optimized by combining the polypeptide subdomains together in various combinations and assessing which combinations of subdomain regions yields the desired result.

A host organism may be chosen for its commercial usefulness in fermentation processes or ability to be genetically manipulated, for example. Increasing the efficiency of production of a desired product produced by commercially useful organisms (e.g., microorganisms in a fermentation process, for example) can yield beneficial gains in starting material conversion and profitability.

Thus, according to the invention, there is provided a method for the preparation of a library of host cells which comprise an assembled polynucleotide at a target locus, which method comprises:
(a) providing a plurality of polynucleotides comprising two or more polynucleotide subgroups, wherein:
(i) a plurality of polynucleotides in each polynucleotide subgroup comprises sequence encoding a peptide or polypeptide and/or a regulatory sequence;
(ii) a plurality of peptides or polypeptides encoded by, or a plurality of regulatory sequences comprised within each polynucleotide subgroup share an activity and/or function;
(iii) at least one polynucleotide subgroup comprises at least two non-identical polynucleotide species;
(iv) a plurality of polynucleotides of each polynucleotide subgroup comprises sequence enabling homologous recombination with a plurality of polynucleotides from one or more other polynucleotide subgroup; and
(v) a plurality of polynucleotides in two polynucleotide subgroups comprises a nucleotide sequence enabling homologous recombination with a target locus in the host cell; and
(b) assembling the polynucleotides at the target locus by homologous recombination in vivo in host cells,
thereby to generate a library of host cells which comprise an assembled polynucleotide at the target locus.

In the invention, a number of polynucleotide subgroups are provided. The polynucleotide subgroups are such that the polynucleotides in a subgroup are capable of homologous recombination with polynucleotides from one or more other groups. In addition, the polynucleotides from two groups are capable of homologous recombination with a target site in the host cells. Accordingly, the method of the invention allows assembled polynucleotides to be generated which typically each comprise a polynucleotide from each of the subgroups and which are incorporated by homologous recombination at a target locus within a host cell. Critically, the assembled polynucleotides are assembled and targeted to a target locus in vivo in host cells. Typically, no polynucleotides in any subgroup will comprise sequence which is an origin or replication.

Plurality is intended to indicate two or more. In the method of the invention, it is possible that all of the plurality of polynucleotides are capable of homologous recombination, that each member of a polynucleotide subgroup comprises sequence which encodes a peptide/polypeptide or which is a regulatory sequence and that each member of a subgroup shares a activity/function. However, the term “plurality” is intended to indicate that there may be polynucleotides within the plurality of polynucleotides which do not undergo homologous recombination and which do not share a function or activity with the other polynucleotides in the same subgroup.

The method according to the invention involves recombination of polynucleotides with each other and with a target locus. Recombination refers to a process in which a molecule of nucleic acid is broken and then joined to a different one. The recombination process of the invention typically involves the artificial and deliberate recombination of disparate nucleic acid molecules, which may be from the same or different organism, so as to create recombinant nucleic acids.

The method of the invention relies on a combination of homologous recombination and site-specific recombination.

“Homologous recombination” refers to a reaction between nucleotide sequences having corresponding sites containing a similar nucleotide sequence (i.e., homologous sequences) through which the molecules can interact (recombine) to form a new, recombinant nucleic acid sequence. The sites of similar nucleotide sequence are each referred to herein as a “homologous sequence”. Generally, the frequency of homologous recombination increases as the length of the homology sequence increases. Thus, while homologous recombination can occur between two nucleic acid sequences that are less than identical, the recombination frequency (or efficiency) declines as the divergence between the two sequences increases.

Recombination may be accomplished using one homology sequence on each of two molecules to be combined, thereby generating a “single-crossover” recombination product. Alternatively, two homology sequences may be placed on each of two molecules to be recombined. Recombination between two homology sequences on the donor with two homology sequences on the target generates a “double-crossover” recombination product.

The polynucleotides with the polynucleotide subgroups can comprise complementary DNA (cDNA). The polynucleotides can consist essentially of cDNA, which refers to a polynucleotide that includes a DNA sequence that encodes mRNA that encodes a polypeptide, and can include one or more non-coding nucleotide sequences that do not have a promoter or other specific function that regulates the amount of mRNA or polypeptide encoded by the DNA (e.g., one or more flanking sequences brought in from a cloning process). The polynucleotides can consist of cDNA. Complementary DNA can be a native (i.e., wild-type) polynucleotide from an organism in some embodiments, and can be a codon-optimized or mutated polynucleotide.

A polynucleotide in the invention may also comprise DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Deoxyribonucleotides include deoxyadenosine, deoxycytidine, deoxyguanosine and deoxythymidine. For RNA, the uracil base is uridine.

The polynucleotides in the polynucleotide subgroups suitable for use in the invention may typically be generated by any amplification process known in the art (e.g., PCR, RT-PCR and the like). Nucleic acid amplification may be particularly beneficial when using organisms that are typically difficult to culture (e.g., slow growing, require specialize culture conditions and the like). The terms “amplify”, “amplification”, “amplification reaction”, or “amplifying” as used herein refer to any in vitro processes for multiplying the copies of a target sequence of nucleic acid. Amplification sometimes refers to an “exponential” increase in target nucleic acid. However, “amplifying” as used herein can also refer to linear increases in the numbers of a select target sequence of nucleic acid, but is different than a one-time, single primer extension step. In some embodiments, a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may also limit inaccuracies associated with depleted reactants in standard PCR reactions. In some embodiments, amplification and/or PCR can be used to add linkers or “sticky-ends” to nucleotide sequences in a combinatorial library to facilitate assembly of combinatorial pathways and/or facilitate inserting assembled pathways into expression constructions of nucleic acid reagents. In some embodiments, a nucleic acid reagent sometimes is stably integrated into the chromosome of the host organism, or a nucleic acid reagent can be a deletion of a portion of the host chromosome, in certain embodiments (e.g., genetically modified organisms, where alteration of the host genome confers the ability to selectively or preferentially maintain the desired organism carrying the genetic modification). Such nucleic acid reagents (e.g., nucleic acids or genetically modified organisms whose altered genome confers a selectable trait to the organism) can be selected for their ability to guide production of a desired protein or nucleic acid molecule. When desired, the nucleic acid reagent can be altered such that codons encode for (i) the same amino acid, using a different tRNA than that specified in the native sequence, or (ii) a different amino acid than is normal, including unconventional or unnatural amino acids (including detectably labeled amino acids). As described herein, the term “native sequence” refers to an unmodified nucleotide sequence as found in its natural setting (e.g., a nucleotide sequence as found in an organism).

Variation can be introduced into one or more polynucleotide subgroups. That is to say a polynucleotide subgroup may comprise two or more non-identical sequences. Thus, by allowing the polynucleotide subgroups to undergo homologous recombination, variant assembled polynucleotides may be generated. The polynucleotide subgroups are assembled in vivo such that a library of host cells is generated comprising variant assembled polynucleotides.

The host cells may be screened to identify a host cell with a desired property conferred by the assembled polynucleotide comprised within that host cell. For example, an assembled polynucleotide may comprise sequences encoding the various members of a pathway. The method can thus be used to identify variant combinations of the members of the pathway that are give rise to, for example, efficient production of a target product.

The number of subgroups is at least two, for example, three, four, five, six, seven, eight, nine, ten, fifteen, twenty, twenty five, thirty, thirty five, forty, forty five or fifty or more. However, typically, there are about 50 of fewer, such as about 20 or fewer polynucleotide subgroups. The method of the invention is intended to generate assembled host cells comprising polynucleotides comprising one polynucleotide from substantially all of the polynucleotide subgroups.

The number of subgroup species combinations is dependent on the number of activities in a given pathway and the number of organisms from which the pathway in question can be isolated. For example, using a three activity subgroup pathway which is found in three organisms, the number of combinatorial permutations mathematically is 3 raised to the power 3, or 3 cubed (e.g., 3³), or 27 in this example. For a three activity pathway where the activities are isolated from four donor organisms, the number of permutations possible is 3⁴ or 81 possible library combinations.

The number of possible combinations in a library therefore can be represented by the formula (X)^{Y}, in certain embodiments, where X is the number of activity subgroups and Y is the number of forms (e.g., species) from which the activity can be effected.

Polynucleotide species in a subgroup can be selected from the following non-limiting forms: codon-optimized forms of a polynucleotide from an organism species, mutated forms of a polynucleotide from an organism species, and native forms of a polynucleotide from a given organism species, for example.

The formula (X)^{Y} is not always indicative of the number of possible combinations in a library. Different subgroups may include different numbers of possible members (or “variants”). For example, one subgroup may include fewer polynucleotide species than another subgroup. One polynucleotide subgroup may include a certain number of native polynucleotides from different organism species and a certain number of engineered polynucleotides (e.g., mutated, codon-optimized versions), and another subgroup may include a fewer or a greater number of each, for example.

As set out above, each subgroup comprises a population of nucleic acids. At least one of the polynucleotide subgroups comprises at least two or more non-identical nucleic acids. That is to say, in a method of the invention, at least two polynucleotides within at least two polynucleotide subgroups are non-identical.

In this way, variation may be introduced such that a library may be generated. More typically, at least two, three, four, five or more polynucleotide subgroups may comprise at least two polynucleotides which are non-identical. The method may be carried out where all polynucleotide subgroups comprise at least two polynucleotides which are non-identical. However, more preferably, a method of the invention is carried out such that at least two polynucleotides within all of the polynucleotide subgroups, other than the two polynucleotide subgroups comprising a nucleotide sequence enabling homologous recombination with a target locus and any polynucleotide subgroup encoding comprises nucleotide sequence encoding a marker gene, are non-identical.

Two of the polynucleotide groups comprise sequences which allow assembled polynucleotides to be incorporated at a target locus (by homologous recombination). This will often result in some sequence at the target locus being replaced with the assembled sequence. The target locus may be a chromosomal locus, i.e. within the genome of the host cell, or an extra-chromosomal locus, for example a plasmid or an artificial chromosome.

One of the two polynucleotide subgroups comprising sequence allowing incorporation at a target locus will typically comprise polynucleotides which are designed to be located at the 5′ end of an assembled polynucleotide. Accordingly, the other of the two polynucleotide groups comprising sequence allowing incorporation at a target locus will typically comprise polynucleotides which are designed to be located at the 3′ end of an assembled polynucleotide. Thus, one of these two subgroups comprises polynucleotides typically capable of homologous recombination with a “5′” sequence of the target locus and the other subgroup comprises polynucleotides typically capable of homologous recombination with a “3′” sequence of the target locus. These sequences may alternatively be referred to as “upstream” (5′) and “downstream” (3′) sequences.

The two subgroups comprising sequence which is intended to enable homologous recombination of the assembled polynucleotide with the target locus will also comprise sequence which allows homologous recombination with one or more of the other subgroups. However, typically, it will not be possible for the polynucleotides within the two subgroups enabling incorporating at the target locus to recombine with each other.

The two subgroups comprising sequence intended to enable homologous recombination at the target locus may, optionally, also comprise additional sequence, for example a sequence encoding a polypeptide which is a member of a pathway to be optimized using the method of the invention.

Typically, the sequences intended to enable incorporation at the target locus will be invariant within a subgroup.

Each subgroup used in a method of the invention comprises polynucleotides having sequence which encodes a peptide or polypeptide and/or comprises a regulatory sequence. The sequence comprised within the polynucleotides or the resulting peptides/polypeptides are typically related. That it to say, each polynucleotide may comprise sequence or encode a peptide/polypeptide which shares an activity and/or a function. For example, each polynucleotide may encode one or more variants of a given enzyme. Alternatively, each polynucleotide may encode alternative polypeptide having substantially the same function, for example, the encoded polypeptides could be alternative marker genes or comprise alternative versions of regulatory sequence. For example, the subgroup could comprise polynucleotides having alternative promoters which are unrelated at the sequence identity level, but nevertheless have the same function of being promoters.

As set out above, each polypeptide encoded by the polynucleotides of a particular polynucleotide subgroup may have a given activity or annotated activity. Such an activity may be the ability to convert a particular substrate into a particular product. Thus, one polypeptide encoded by a polynucleotide in a subgroup may convert a first substrate to a first product with more efficiency than it converts a second substrate to a second product, yet it has the same activity as another polypeptide in the same subgroup that also converts the second substrate to the second product. For example, (i) one polypeptide in a subgroup may prefer to convert a six-carbon substrate to product, but with less efficiency also will convert a five-carbon substrate to a product, and (ii) another polypeptide in a subgroup may prefer to convert the same five-carbon substrate to same product; these two polypeptides share the same activity of converting the same five-carbon substrate to the same product. An activity may be the ability to bind a particular molecule.

The term “same activity” as used herein refers to substantially the same type of activity (e.g., the ability to convert a certain substrate into a certain product) without regard to the level of activity, or efficiency, so long as the activity is detectable for both polynucleotides (or the polypeptides encoded by those polynucleotides).

Each polypeptide encoded by in a particular polynucleotide subgroup may be able to bind to a particular molecule (e.g., substrate, ligand and the like).

Polynucleotides or polypeptides encoded by such polynucleotides in a particular subgroup may share at least about 60% nucleic acid or amino acid sequence identity. That is, polynucleotides or polypeptides in or encoded by a particular polynucleotide subgroup can share about 61% or greater, 62% or greater, 63% or greater, 64% or greater, 65% or greater, 66% or greater, 67% or greater, 68% or greater, 69% or greater, 70% or greater, 71% or greater, 72% or greater, 73% or greater, 74% or greater, 75% or greater, 76% or greater, 77% or greater, 78% or greater, 79% or greater, 80% or greater, 81% or greater, 82% or greater, 83% or greater, 84% or greater, 85% or greater, 86% or greater, 87% or greater, 88% or greater, 89% or greater, 90% or greater, 91% or greater, 92% or greater, 93% or greater, 94% or greater, 95% or greater, 96% or greater, 97% or greater, 98% or greater, 99% or greater nucleic acid or amino acid sequence identity.

Two polypeptides encoded by a polynucleotide subgroup may have a different activity when they each convert a different substrate into a product (e.g., a different or same product), or convert the same substrate into a different product. Two polypeptides can bind to a different molecule (e.g., substrate, ligand) and have a different activity. Two polypeptides having a different activity typically do not share a common activity.

Polynucleotides or polypeptides encoded by polynucleotides in different subgroups may share a common activity. More typically, however, polynucleotides/polypeptides in different subgroups do not share a common activity. That is to say, the peptides or polypeptides encoded by or regulatory sequence comprised within a given polynucleotide subgroup may have a different activity and/or function than those of every other polynucleotide subgroup.

Polypeptides encoded by polypeptides in different subgroups may share a common secondary activity, for example a common activity in a pathway being optimized or a common side-activity.

The invention may be used to optimize a pathway in the sense that is may be used to identify the optimal activities to carry out a biochemical transformation, wherein the precise sequence of steps may or may not be known. For example, cellulosic degradation is believed to require the activity of a number of related enzymes. The method of the invention may be used to determine optimal combinations of such related enzymes. Different polynucleotide subgroups used in the invention would, in the case, typically encode variants of such related enzymes. Exocellulase which cleave two to four units from the ends of exposed chains produced by endocellulase, resulting in the tetrasaccharides or disaccharides, such as cellobiose are important in cellulose degradation. There are two main types of exocellulases [or cellobiohydrolases (CBH)]—CBHI works processively from the reducing end, and CBHII works processively from the nonreducing end of cellulose. For the purposes of the invention, and by way of example, CBHI and CBHII may be considered to have different activity, i.e. would typically be comprised within different polynucleotide subgroups, although they are both exocellulases. Thus, the invention could be used to identify more optimal combinations of CBHI and CBHII variants. A single polynucleotide subgroup may though comprise sequences encoding CBHI and CBHII variants in the context of identifying combinations of exocellulases with other cellulose degrading enzymes.

For the purposes of this invention, activity may be ascribed on the basis of, for example, known biochemical activity or annotation based on bio-informatic analysis.

Each activity may be carried out by a polypeptide encoded by polynucleotide. The polynucleotides used in the invention may comprise complementary DNA (cDNA). The polynucleotides used in the invention may consist essentially of cDNA. A cDNA may encode mRNA that in turn encodes a polypeptide. Thus, each activity subgroup can be represented by a polynucleotide subgroup that encodes a polypeptide having a particular activity. The activity of a peptide or polypeptide may optionally be apparent only after processing. For example, several enzymes are functional only when further processing, such as cleavage, phosphorylation, has taken place.

In the method of the invention, each polynucleotide in at least one polynucleotide subgroup may comprise nucleotide sequence encoding a marker gene. Typically, each polynucleotide will encode the same marker gene. However, the method may be carried out where two or more different marker genes are encoded by the polynucleotides within the subgroup. The marker gene may be used to identify those host cells into which an assembled polynucleotide has been incorporated.

Any suitable marker gene may be used and such genes are well known to determine whether a nucleic acid is included in a cell. An assembled polynucleotide prepared according to the invention may comprise two or more marker genes, where one functions efficiently in one organism and another functions efficiently in another organism.

Examples of marker genes include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotic resistance markers (e.g., β-lactamase), β-galactosidase, fluorescent or other coloured markers, such as green fluorescent protein (GFP), yellow fluorescent protein (YFP), red fluorescent protein (RFP) and cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments as described in 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

The method of the invention is typically used to generate library of host cells, wherein each host cell harbours at least one assembled polynucleotide at one or more target loci.

The polynucleotide subgroups are introduced into host cells so as to generate such libraries. The polynucleotide subgroups can be introduced into host cells using various techniques. Non-limiting examples of methods used to introduce heterologous nucleic acids into various organisms include; transformation, transfection, transduction, electroporation, ultrasound-mediated transformation, particle bombardment and the like. In some instances the addition of carrier molecules can increase the uptake of DNA in cells typically though to be difficult to transform by conventional methods. Conventional methods of transformation are readily available to the skilled person.

The method can be used to generate a library of host cells, wherein at least about 50% of the host cells in the library comprise an assembled polynucleotide which comprises one polynucleotide from each polynucleotide subgroup. The method may be used to generate a library of host cells, wherein at least about 50%, for example at least about 60%, at least about 70%, at least about 80%, at least about 85%, at least about 90%, at least about 95%, at least about 96%, at least about 97%, at least about 98%, at least about 99%, of host cells harbour at least one assembled polynucleotide at one or more target loci.

A host cell library generated according to the invention can comprise at least about 20 to at least about 1,000,000 different assembled polynucleotides, for example at least about 100, at least about 1,000, at least about 10,000, at least about 100,000, at least about 500,000 variant assembled polynucleotides.

There may be multiple copy numbers of each assembled polynucleotide in a library prepared according to the method of the invention. Generally, an individual host cell within such a library can include one. However, an individual host cell may include two or more nucleic acid species. Individual host cells may be isolated and tested for target product production, and an individual host cell may be proliferated after isolation and before testing.

A host cell library generated according to the invention can comprise assembled polypeptides having substantially all possible combinations of subgroup polynucleotides. The method of the invention may be used to generate a library of host cells that includes at least about 60% of all possible subgroup polynucleotide combinations (e.g., about 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more of all possible subgroup species combinations).

In the method of the invention, generally at least one assembled polynucleotide will comprise each member of a biological pathway. Preferably, the biological pathway enables the production of a compound of interest in the host cell.

In the method of the invention, each assembled polynucleotide may include one polynucleotide species from each of the plurality of polynucleotide subgroups. Each assembled polynucleotide may include more than one polynucleotide subgroup from a given donor organism. That is to say, in a pathway that has multiple activities, an optimized pathway may comprise more than one polynucleotide subgroup from a given donor organism. The polynucleotides within a polynucleotide subgroup can be from a different donor organism type, where a different “type” can refer to a different genus, species, or strain, for example.

Each assembled polynucleotide may comprise polynucleotide species linked in series. The polynucleotide species may be separated from one another by linkers.

The compound of interest may a primary metabolite, secondary metabolite, a peptide or polypeptide or it may include biomass comprising the host cell itself. The compounds of interest may be an organic compound selected from glucaric acid, gluconic acid, glutaric acid, adipic acid, succinic acid, tartaric acid, oxalic acid, acetic acid, lactic acid, formic acid, malic acid, maleic acid, malonic acid, citric acid, fumaric acid, itaconic acid, levulinic acid, xylonic acid, aconitic acid, ascorbic acid, kojic acid, comeric acid, an amino acid, a poly unsaturated fatty acid, ethanol, 1,3-propane-diol, ethylene, glycerol, xylitol, carotene, astaxanthin, lycopene and lutein. Alternatively, the fermentation product may be a β-lactam antibiotic such as Penicillin G or Penicillin V and fermentative derivatives thereof, a cephalosporin, cyclosporin or lovastatin.

The compound of interest may be a peptide selected from an oligopeptide, a polypeptide, a (pharmaceutical or industrial) protein and an enzyme. In such processes the peptide is preferably secreted from the host cell, more preferably secreted into the culture medium such that the peptide may easily be recovered by separation of the host cellular biomass and culture medium comprising the peptide, e.g. by centrifugation or (ultra)filtration.

Examples of proteins or (poly)peptides with industrial applications that may be produced in the methods of the invention include enzymes such as e.g. lipases (e.g. used in the detergent industry), proteases (used inter alia in the detergent industry, in brewing and the like), carbohydrases and cell wall degrading enzymes (such as, amylases, glucosidases, cellulases, pectinases, beta-1,3/4- and beta-1,6-glucanases, rhamnogalacturonases, mannanases, xylanases, pullulanases, galactanases, esterases and the like, used in fruit processing, wine making and the like or in feed), phytases, phospholipases, glycosidases (such as amylases, beta.-glucosidases, arabinofuranosidases, rhamnosidases, apiosidases and the like), dairy enzymes and products (e.g. chymosin, casein), polypeptides (e.g. poly-lysine and the like, cyanophycin and its derivatives). Mammalian, and preferably human, polypeptides with therapeutic, cosmetic or diagnostic applications include, but are not limited to, collagen and gelatin, insulin, serum albumin (HSA), lactoferrin and immunoglobulins, including fragments thereof. The polypeptide may be an antibody or a part thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase.

In the method of the invention, one or more polynucleotide subgroups will typically comprise polynucleotides having sequence encoding variants of a polypeptide or comprise variants of a regulatory sequence.

The variants may be members of a gene cluster. A gene cluster is a set of two or more genes that serve to encode for the same or similar products. An example of a gene cluster is the human β-globin gene cluster, which contains five functional genes and one non-functional gene which code for similar proteins. Hemoglobin molecules contain any two identical proteins from this gene cluster, depending on their specific role.

The variants may be allelic or species variants of a polypeptide or regulatory sequence.

The variants may be artificial variants.

The variants may share at least about 40% sequence identity with each other. However, the variants may share at least about 50%, at least about 60%, at least about 60%, at least about 60%, at least about 60%, at least about 65%, at least about 70%, at least about 75%, at least about 80%, at least about 85%, at least about 90%, at least about at least about 95%, at least about 96%, at least about 97%, at least about 98% or at least about 99% sequence identity.

Sequence identity may be calculated at the level of the polynucleotide or at the level of the polypeptide encoded by the polynucleotide variants. Methods for determining sequence identity are described herein. Such identity is intended to be determined across the length of the variants concerned, not the entire length of the polynucleotide of which the variant may be a part.

Variant sequences may be prepared by isolation or amplification from a suitable source without any further modification. However, polynucleotides prepared by isolation or amplification may be genetically modified to generate additional variants, typically with the aim of altering (e.g., increase or decrease, for example) the activity of polypeptide encoded by the polynucleotide.

In some embodiments, nucleic acids, used to add an activity to an organism, sometimes are genetically modified to optimize the heterologous polynucleotide sequence encoding the desired activity (e.g., polypeptide or protein, for example). The term “optimize” as used herein can refer to alteration to increase or enhance expression by preferred codon usage. The term optimize can also refer to modifications to the amino acid sequence to increase the activity of a polypeptide or protein, such that the activity exhibits a higher catalytic activity as compared to the “natural” version of the polypeptide or protein.

Nucleotide sequences of interest can be genetically modified using methods known in the art. Mutagenesis techniques are particularly useful for small scale (e.g., 1, 2, 5, 10 or more nucleotides) or large scale (e.g., 50, 100, 150, 200, 500, or more nucleotides) genetic modification. Mutagenesis allows the artisan to alter the genetic information of an organism in a stable manner, either naturally (e.g., isolation using selection and screening) or experimentally by the use of chemicals, radiation or inaccurate DNA replication (e.g., PCR mutagenesis). In some embodiments, genetic modification can be performed by whole scale synthetic synthesis of nucleic acids, using a native nucleotide sequence as the reference sequence, and modifying nucleotides that can result in the desired alteration of activity. Mutagenesis methods sometimes are specific or targeted to specific regions or nucleotides (e.g., site-directed mutagenesis, PCR-based site-directed mutagenesis, and in vitro mutagenesis techniques such as transplacement and in vivo oligonucleotide site-directed mutagenesis, for example). Mutagenesis methods sometimes are non-specific or random with respect to the placement of genetic modifications (e.g., chemical mutagenesis, insertion element (e.g., insertion or transposon elements) and inaccurate PCR based methods, for example).

In some embodiments, an ORF nucleotide sequence sometimes is mutated or modified to alter the triplet nucleotide sequences used to encode amino acids (e.g., amino acid codon triplets, for example). Modification of the nucleotide sequence of an ORF to alter codon triplets sometimes is used to change the codon found in the original sequence to better match the preferred codon usage of the organism in which the ORF or nucleic acid reagent will be expressed. For example, the codon usage, and therefore the codon triplets encoded by a nucleotide sequence from bacteria may be different from the preferred codon usage in eukaryotes like yeast or plants. Preferred codon usage also may be different between bacterial species. In certain embodiments an ORF nucleotide sequences sometimes is modified to eliminate codon pairs and/or eliminate mRNA secondary structures that can cause pauses during translation of the mRNA encoded by the ORF nucleotide sequence. Translational pausing sometimes occurs when nucleic acid secondary structures exist in an mRNA, and sometimes occurs due to the presence of codon pairs that slow the rate of translation by causing ribosomes to pause. In some embodiments, the use of lower abundance codon triplets can reduce translational pausing due to a decrease in the pause time needed to load a charged tRNA into the ribosome translation machinery. Therefore, to increase transcriptional and translational efficiency in bacteria (e.g., where transcription and translation are concurrent, for example) or to increase translational efficiency in eukaryotes (e.g., where transcription and translation are functionally separated), the nucleotide sequence of a nucleotide sequence of interest can be altered to better suit the transcription and/or translational machinery of the host and/or genetically modified microorganism. In certain embodiments, slowing the rate of translation by the use of lower abundance codons, which slow or pause the ribosome, can lead to higher yields of the desired product due to an increase in correctly folded proteins and a reduction in the formation of inclusion bodies.

Codons can be altered and optimized according to the preferred usage by a given organism by determining the codon distribution of the nucleotide sequence donor organism and comparing the distribution of codons to the distribution of codons in the recipient or host organism. Techniques described herein (e.g., site directed mutagenesis and the like) can then be used to alter the codons accordingly.

Comparisons of codon usage can be done by hand, or using nucleic acid analysis software commercially available to the artisan. Modification of the nucleotide sequence of an ORF also can be used to correct codon triplet sequences that have diverged in different organisms. For example, certain yeast (e.g., C. tropicalis and C. maltosa) use the amino acid triplet CUG (e.g., CTG in the DNA sequence) to encode serine. CUG typically encodes leucine in most organisms. In order to maintain the correct amino acid in the resultant polypeptide or protein, the CUG codon must be altered to reflect the organism in which the nucleic acid reagent will be expressed. Thus, if an ORF from a bacterial donor is to be expressed in either Candida yeast strain mentioned above, the heterologous nucleotide sequence must first be altered or modified to the appropriate leucine codon. Therefore, in some embodiments, the nucleotide sequence of an ORF sometimes is altered or modified to correct for differences that have occurred in the evolution of the amino acid codon triplets between different organisms. In some embodiments, the nucleotide sequence can be left unchanged at a particular amino acid codon, if the amino acid encoded is a conservative or neutral change in amino acid when compared to the originally encoded amino acid.

Site directed mutagenesis is a procedure in which a specific nucleotide or specific nucleotides in a DNA molecule are mutated or altered. Site directed mutagenesis typically is performed using a nucleotide sequence of interest cloned into a circular plasmid vector. Site-directed mutagenesis requires that the wild type sequence be known and used a platform for the genetic alteration. Site-directed mutagenesis sometimes is referred to as oligonucleotide-directed mutagenesis because the technique can be performed using oligonucleotides which have the desired genetic modification incorporated into the complement a nucleotide sequence of interest. The wild type sequence and the altered nucleotide are allowed to hybridize and the hybridized nucleic acids are extended and replicated using a DNA polymerase. The double stranded nucleic acids are introduced into a host (e.g., E. coli, for example) and further rounds of replication are carried out in vivo. The transformed cells carrying the mutated nucleotide sequence are then selected and/or screened for those cells carrying the correctly mutagenized sequence. Cassette mutagenesis and PCR-based site-directed mutagenesis are further modifications of the site-directed mutagenesis technique. Site-directed mutagenesis can also be performed in vivo (e.g., transplacement “pop-in pop-out”, In vivo site-directed mutagenesis with synthetic oligonucleotides and the like, for example).

PCR-based mutagenesis can be performed using PCR with oligonucleotide primers that contain the desired mutation or mutations. The technique functions in a manner similar to standard site-directed mutagenesis, with the exception that a thermocycler and PCR conditions are used to replace replication and selection of the clones in a microorganism host. As PCR-based mutagenesis also uses a circular plasmid vector, the amplified fragment (e.g., linear nucleic acid molecule) containing the incorporated genetic modifications can be separated from the plasmid containing the template sequence after a sufficient number of rounds of thermocycler amplification, using standard electrophorectic procedures. A modification of this method uses linear amplification methods and a pair of mutagenic primers that amplify the entire plasmid. The procedure takes advantage of the E. coli Dam methylase system which causes DNA replicated in vivo to be sensitive to the restriction endonucleases Dpnl. PCR synthesized DNA is not methylated and is therefore resistant to Dpnl. This approach allows the template plasmid to be digested, leaving the genetically modified, PCR synthesized plasmids to be isolated and transformed into a host bacteria for DNA repair and replication, thereby facilitating subsequent cloning and identification steps. A certain amount of randomness can be added to PCR-based sited directed mutagenesis by using partially degenerate primers.

Chemical mutagenesis often involves chemicals like ethyl methanesulfonate (EMS), nitrous acid, mitomycin C, N-methyl-N-nitrosourea (MNU), diepoxybutane (DEB), 1,2,7,8-diepoxyoctane (DEO), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl̂-chloroethylj-aminopropylaminôacridinedihydrochloride (ICR-170), 2-amino purine (2AP), and hydroxylamine (HA), provided herein as non-limiting examples. These chemicals can cause base-pair substitutions, frameshift mutations, deletions, transversion mutations, transition mutations, incorrect replication, and the like. In some embodiments, the mutagenesis can be carried out in vivo. Sometimes the mutagenic process involves the use of the host organisms DNA replication and repair mechanisms to incorporate and replicate the mutagenized base or bases.

Another type of chemical mutagenesis involves the use of base-analogs. The use of base-analogs cause incorrect base pairing which in the following round of replication is corrected to a mismatched nucleotide when compared to the starting sequence. Base analog mutagenesis introduces a small amount of non-randomness to random mutagenesis, because specific base analogs can be chose which can be incorporated at certain nucleotides in the starting sequence. Correction of the mispairing typically yields a known substitution. For example, Bromo-deoxyuridine (BrdU) can be incorporated into DNA and replaces T in the sequence. The host DNA repair and replication machinery can sometime correct the defect, but sometimes will mispair the BrdU with a G. The next round of replication then causes a G-C transversion from the original A-T in the native sequence. Ultra violet (UV) induced mutagenesis is caused by the formation of thymidine dimers when UV light irradiates chemical bonds between two adjacent thymine residues. Excision repair mechanism of the host organism correct the lesion in the DNA, but occasionally the lesion is incorrectly repaired typically resulting in a C to T transition.

DNA shuffling is a method which uses DNA fragments from members of a mutant library and reshuffles the fragments randomly to generate new mutant sequence combinations. The fragments are typically generated using DNasel, followed by random annealing and re-joining using self priming PCR. The DNA overhanging ends, from annealing of random fragments, provide “primer” sequences for the PCR process. Shuffling can be applied to libraries generated by any of the above mutagenesis methods. Error prone PCR and its derivative rolling circle error prone PCR uses increased magnesium and manganese concentrations in conjunction with limiting amounts of one or two nucleotides to reduce the fidelity of the Taq polymerase. The error rate can be as high as 2% under appropriate conditions, when the resultant mutant sequence is compared to the wild type starting sequence. After amplification, the library of mutant coding sequences must be cloned into a suitable plasmid. Although point mutations are the most common types of mutation in error prone PCR, deletions and frameshift mutations are also possible. There are a number of commercial error-prone PCR kits available, including those from Stratagene and Clontech (e.g., World Wide Web URL strategene.com and World Wide Web URL clontech.com, respectively, for example). Rolling circle error-prone PCR is a variant of error-prone PCR in which wild-type sequence is first cloned into a plasmid, the whole plasmid is then amplified under error-prone conditions. As noted above, organisms with altered activities can also be isolated using genetic selection and screening of organisms challenged on selective media or by identifying naturally occurring variants from unique environments. For example, 2-Deoxy-D-glucose is a toxic glucose analog. Growth of yeast on this substance yields mutants that are glucose-deregulated. A number of mutants have been isolated using 2-Deoxy-D-glucose including transport mutants, and mutants that ferment glucose and galactose simultaneously instead of glucose first then galactose when glucose is depleted. Similar techniques have been used to isolate mutant microorganisms that can metabolize plastics (e.g., from landfills), petrochemicals (e.g., from oil spills), and the like, either in a laboratory setting or from unique environments.

Thus, the activity of a polynucleotide can be altered by modifying the nucleotide sequence of a coding sequence, for example, by point mutation, deletion mutation, insertion mutation, PCR based mutagenesis and the like) to alter, enhance or increase, reduce, substantially reduce or eliminate the activity of the encoded protein or peptide. The protein or peptide encoded by a modified coding sequence sometimes is produced in a lower amount or may not be produced at detectable levels, and in other embodiments, the product or protein encoded by the modified coding sequence is produced at a higher level (e.g., codons sometimes are modified so they are compatible with tRNA's preferentially used in the host organism or engineered organism). To determine the relative activity, the activity from the product of the mutated ORF (or cell containing it) can be compared to the activity of the product or protein encoded by the unmodified ORF (or cell containing it).

In the method of the invention, a plurality of polynucleotides in each polynucleotide subgroup comprises sequence encoding a peptide or polypeptide and/or a regulatory sequence. Thus, a polynucleotide in a subgroup may comprise one or more of, for example: a promoter element, an enhancer element, a 5′ untranslated region (5′ UTR) or 3′ untranslated region (3′ UTR). These elements may be present where there is no coding sequence. Alternatively, they may be operably linked with a coding sequence also present on the polynucleotide.

Accordingly, a polynucleotide subgroup may comprise regulatory element and/or a coding sequence. Thus, the method of the invention may be used to determine, for example, the best promoter for use in connection with a given coding sequence. Thus, one polynucleotide subgroup may comprise a promoter and the “adjacent” subgroup (in the sense that it will be immediately 3′ to the promoter subgroup in the assembled polynucleotide) may comprise a coding sequence. In this way, optimal combinations of promoter and coding sequence may be determined. This approach may further be combined with additional subgroups in which the polynucleotides comprise, for example 5′ and 3′ UTRs.

A promoter element typically is required for DNA synthesis and/or RNA synthesis. A promoter element often comprises a region of DNA that can facilitate the transcription of a particular gene, by providing a start site for the synthesis of RNA corresponding to a gene. Promoters generally are located near the genes they regulate, are located upstream of the gene (e.g., 5′ of the gene), and are on the same strand of DNA as the sense strand of the gene, in some embodiments.

A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the chosen expression system (e.g., expression in a chosen organism, or expression in a cell free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, −35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5′ UTR in a polynucleotide subgroup can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 4OS ribosome binding sequence) and sometimes is an internal ribosome entry sequence (IRES). An IRES generally forms an RNA scaffold with precisely placed RNA tertiary structures that contact a 4OS ribosomal subunit via a number of specific intermolecular interactions. Examples of ribosomal enhancer sequences are known and can be identified by the skilled person (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); GalMe, Nucleic Acids Research 30: 3401-341 1 (2002); Shaloiko et al., http address www.interscience.wiley.com, DOI: 10.1002/bit.20267; and Gallie et al., Nucleic Acids Research 15: 3257-3273 (1987)). A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25 nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The skilled person can select appropriate elements for the 3′ UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted). In some embodiments, modification of a 5′ UTR and/or a 3′ UTR can be used to alter (e.g., increase, add, decrease or substantially eliminate) the activity of a promoter.

In a method of the invention, each polynucleotide within a subgroup encoding a polypeptide may be operably linked with a promoter. However, each polynucleotide within the same subgroup may not necessarily be in operable linkage with the same promoter. Thus, a subgroup may comprise polynucleotides having different promoters.

The polynucleotide species may thus be in operable linkage with one or more promoters. Polypeptide-encoding polynucleotides in different subgroups may be in operable linkage with separate promoters. Thus, an assembled polynucleotide may include a specific promoter operably for each polynucleotide subgroup (e.g., for an assembled nucleic acid containing a polynucleotide from each of six polynucleotide subgroups, there will typically be six promoter present, where each promoter is operably linked to each constituent polynucleotide of the assembled polynucleotide). In some embodiments, a promoter operably linked to a polynucleotide nucleotide may be the same or different for two or more polynucleotide subgroups represented within an assembled polynucleotide. For example, in an assembled polynucleotide containing a polynucleotide from each of six polynucleotide subgroups, there can be six promoters, each operably linked to a polynucleotide, where (i) all promoters are the same, (ii) all promoters are different, (iii) some promoters are the same and some promoters are different (e.g., 2 promoters are the same and 4 promoters are different).

In the method of the invention, the polynucleotides within the polynucleotide subgroups may be from about 50 bp to about 10 kb in length.

In the method of the invention, the sequences enabling homologous recombination may be from about 20 bp to about 500 kb in length.

In order to promote targeted integration at a targeted locus and to ensure assembly of the polynucleotide subgroups: (i) each polynucleotide of each polynucleotide subgroup comprises sequence enabling homologous recombination with each polynucleotide from one or more other polynucleotide subgroup; and (ii) each polynucleotide in two polynucleotide subgroups comprises sequence enabling homologous recombination with a target sequence in the host cell.

Homologous recombination is a type of genetic recombination in which nucleotide sequences are exchanged between two similar or identical molecules of DNA. The lengths of the sequences mediating homologous recombination between polynucleotide subgroups and with the target locus may be at least about 20 bp, at least about 30 bp, at least about 50 bp, at least about 0.1 kb, at least about 0.2 kb, at least about 0.5 kb, at least about 1 kb or at least about 2 kb.

As set out above, in the method of the invention, the assembled polynucleotide may be recombined at a target locus in the genome of the host cells, for example at a chromosomal location, or into an extra-chromosomal target locus. The target locus may be any suitable locus within the genome of the host cell. The extra-chromosomal target locus may be a plasmid or an artificial chromosome, such as a yeast artificial chromosome, for example where the host cells are yeast cells.

Recombination of the assembled polynucleotide at a target locus may result in insertion of the assembled polynucleotide at the target locus such that no genetic material is lost at the locus (although the assembled polynucleotide will disrupt the locus). However, recombination of the assembled polynucleotide at a target locus may replace genetic material at the target locus.

The polynucleotides in one or more polynucleotide subgroups may comprise one or more site-specific recombinase sites, for example, so that an assembled polynucleotide may be recovered from a host cell. A site-specific recombinase insertion site is a recognition sequence on a nucleic acid molecule that participates in an integration/recombination reaction by recombination proteins such as Cre recombinase. The site recognized by Cre recombinase is loxP, which is a 34 base pair sequence comprised of two 13 base pair inverted repeats (serving as the recombinase binding sites) flanking an 8 base pair core sequence. Other examples of recombination sites include attB, attP, attL, and attR sequences, and mutants, fragments, variants and derivatives thereof, which are recognized by the recombination protein λInt and by the auxiliary proteins integration host factor (IHF), FIS and excisionase (Xis).

Conveniently, such sites may be located in the polynucleotide subgroups comprising sequences which enable homologous recombination with the target locus. In that way, the entire assembled polynucleotide may, conveniently, be recovered from a host cell.

In the method of the invention, the host cells are typically those of an organism suitable for genetic manipulation and one which may be cultured at cell densities useful for industrial production of a target product. A suitable organism may be a microorganism, for example one which may be maintained in a fermentation device.

A host cell may be a prokaryotic, archaebacterial or eukaryotic organism, or a cell form such an organism.

A host cell suitable for use in the invention can include one or more of the following features: aerobe, anaerobe, filamentous, non-filamentous, monoploid, dipoid, auxotrophic and/or non-auxotrophic.

A host cell suitable for use in the invention may be a prokaryotic microorganism (e.g., bacterium) or a non-prokaryotic microorganism. A suitable host cell may be a eukaryotic microorganism (e.g., yeast, fungi, amoeba, and algae). A suitable host cell may be from a non-microbial source, for example a mammalian or insect cell.

“Fungi” are herein defined as eukaryotic microorganisms and include all species of the subdivision Eumycotina (Alexopoulos, C. J., 1962, In: Introductory Mycology, John Wiley & Sons, Inc., New York). The term fungus thus includes both filamentous fungi and yeast. “Filamentous fungi” are herein defined as eukaryotic microorganisms that include all filamentous forms of the subdivision Eumycotina and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, and Trichoderma.

“Yeasts” are herein defined as eukaryotic microorganisms and include all species of the subdivision Eumycotina that predominantly grow in unicellular form. Yeasts may either grow by budding of a unicellular thallus or may grow by fission of the organism.

The host cells according to the invention are preferably fungal host cell whereby a fungus is defined as herein above. Preferred fungal host cells are fungi that are used in industrial fermentation processes for the production of fermentation products as described below. A large variety of filamentous fungi as well as yeasts are use in such processes. Preferred filamentous fungal host cells may be selected from the genera: Aspergillus, Trichoderma, Humicola, Acremonium, Fusarium, Rhizopus, Mortierella, Penicillium, Myceliophthora, Chrysosporium, Mucor, Sordaria, Neurospora, Podospora, Monascus, Agaricus, Pycnoporus, Schizophylum, Trametes and Phanerochaete. Preferred fungal strains that may serve as host cells, e.g. as reference host cells for the comparison of fermentation characteristics of transformed and untransformed cells, include e.g. Aspergillus niger CBS120.49, CBS 513.88, Aspergillus oryzae ATCC16868, ATCC 20423, IFO 4177, ATCC 1011, ATCC 9576, ATCC14488-14491, ATCC 11601, ATCC12892, Aspergillus fumigatus AF293 (CBS101355), P. chrysogenum CBS 455.95, Penicillium citrinum ATCC 38065, Penicillium chrysogenum P2, Acremonium chrysogenum ATCC 36225, ATCC 48272, Trichoderma reesei ATCC 26921, ATCC 56765, ATCC 26921, Aspergillus sojae ATCC11906, Chrysosporium lucknowense ATCC44006 and derivatives of all of these strains. Particularly preferred as filamentous fungal host cell are Aspergillus niger CBS 513.88 and derivatives thereof.

Any suitable yeast may be selected as a host cell. Preferred yeast host cells may be selected from the genera: Saccharomyces (e.g., S. cerevisiae, S. bayanus, S. pastorianus, S. carlsbergensis), Kluyveromyces, Candida (e.g., C. revkaufi, C. pulcherrima, C. tropicalis, C. utilis), Pichia (e.g., P. pastoris), Schizosaccharomyces, Hansenula, Kloeckera, Schwanniomyces, and Yarrowia (e.g., Y. lipolytica (formerly classified as Candida lipolytica)).

Any suitable prokaryote may be selected as a host cell. A Gram negative or Gram positive bacteria may be selected. Examples of bacteria include, but are not limited to, Bacillus bacteria (e.g., B. subtilis, B. megaterium), Acinetobacter bacteria, Norcardia baceteria, Xanthobacter bacteria, Escherichia bacteria (e.g., E. coli (e.g., strains DH 1 OB, Stbl2, DH5-alpha, DB3, DB3.1), DB4, DB5, JDP682 and ccdA-over (e.g., U.S. application Ser. No. 09/518,188))), Streptomyces bacteria, Erwinia bacteria, Klebsiella bacteria, Serratia bacteria (e.g., S. marcessans), Pseudomonas bacteria (e.g., P. aeruginosa), Salmonella bacteria (e.g., S. typhimurium, S. typhi). Bacteria also include, but are not limited to, photosynthetic bacteria (e.g., green non-sulfur bacteria (e.g., Choroflexus bacteria (e.g., C. aurantiacus), Chloronema bacteria (e.g., C. gigateum)), green sulfur bacteria (e.g., Chlorobium bacteria (e.g., C. limicola), Pelodictyon bacteria (e.g., P. luteolum), purple sulfur bacteria (e.g., Chromatium bacteria (e.g., C. okenii)), and purple non-sulfur bacteria (e.g., Rhodospirillum bacteria (e.g., R. rubrum), Rhodobacter bacteria (e.g., R. sphaeroides, R. capsulatus), and Rhodomicrobium bacteria (e.g., R. vanellii)).

Cells from non-microbial organisms can be utilized as a host cell. Examples of such cells, include, but are not limited to, insect cells (e.g., Drosophila (e.g., D. melanogaster), Spodoptera (e.g., S. frugiperda Sf9 or Sf21 cells) and Trichoplusa (e.g., High-Five cells); nematode cells (e.g., C. elegans cells); avian cells; amphibian cells (e.g., Xenopus laevis cells); reptilian cells; and mammalian cells (e.g., NIH3T3, 293, CHO, COS, VERO, C127, BHK, Per-C6, Bowes melanoma and HeLa cells).

Microorganisms or cells suitable for use as host cells in the invention are commercially available.

Eukaryotic cells have at least two separate pathways (one via homologous recombination (HR) and one via non-homologous recombination (NHR)) through which nucleic acids (in particular DNA) can be integrated into the host genome. The yeast Saccharomyces cerevisiae is an organism with a preference for homologous recombination (HR). The ratio of non-homologous to homologous recombination (NHR/HR) of this organism may vary from about 0.07 to 0.007.

WO 02/052026 discloses mutants of S. cerevisiae having an improved targeting efficiency of DNA sequences into its genome. Such mutant strains are deficient in a gene involved in NHR (KU70).

Contrary to S. cerevisiae, most higher eukaryotes such as filamentous fungal cells up to mammalian cells have a preference for NHR. Among filamentous fungi, the NHR/HR ratio ranges between 1 and more than 100. In such organisms, targeted integration frequency is rather low.

Thus, to improve the efficiency of polynucleotide assembly at the target locus, it is preferred that the efficiency of homologous recombination (HR) is enhanced in the host cell in the method according to the invention.

Accordingly, preferably in the method according to the invention, the host cell is, preferably inducibly, increased in its efficiency of homologous recombination (HR). Since the NHR and HR pathways are interlinked, the efficiency of HR can be increased by modulation of either one or both pathways. Increase of expression of HR components will increase the efficiency of HR and decrease the ratio of NHR/HR. Decrease of expression of NHR components will also decrease the ratio of NHR/HR The increase in efficiency of HR in the host cell of the vector-host system according to the invention is preferably depicted as a decrease in ratio of NHR/HR and is preferably calculated relative to a parent host cell wherein the HR and/or NHR pathways are not modulated. The efficiency of both HR and NHR can be measured by various methods available to the person skilled in the art. A preferred method comprises determining the efficiency of targeted integration and ectopic integration of a single vector construct in both parent and modulated host cell. The ratio of NHR/HR can then be calculated for both cell types. Subsequently, the decrease in NHR/HR ration can be calculated. In WO2005/095624, this preferred method is extensively described.

Host cells having a decreased NHR/HR ratio as compared to a parent cell may be obtained by modifying the parent eukaryotic cell by increasing the efficiency of the HR pathway and/or by decreasing the efficiency of the NHR pathway. Preferably, the NHR/HR ratio thereby is decreased at least twice, preferably at least 4 times, more preferably at least 10 times. Preferably, the NHR/HR ratio is decreased in the host cell of the vector-host system according to the invention as compared to a parent host cell by at least 5%, more preferably at least 10%, even more preferably at least 20%, even more preferably at least 30%, even more preferably at least 40%, even more preferably at least 50%, even more preferably at least 60%, even more preferably at least 70%, even more preferably at least 80%, even more preferably at least 90% and most preferably by at least 100%.

According to one embodiment, the ratio of NHR/HR is decreased by increasing the expression level of an HR component. HR components are well-known to the person skilled in the art. HR components are herein defined as all genes and elements being involved in the control of the targeted integration of polynucleotides into the genome of a host, said polynucleotides having a certain homology with a certain pre-determined site of the genome of a host wherein the integration is targeted.

The ratio of NHR/HR may be decreased by decreasing the expression level of an NHR component. NHR components are herein defined as all genes and elements being involved in the control of the integration of polynucleotides into the genome of a host, irrespective of the degree of homology of said polynucleotides with the genome sequence of the host. NHR components are well-known to the person skilled in the art. Preferred NHR components are a component selected from the group consisting of the homolog or ortholog for the host cell of the vector-host system according to the invention of the yeast genes involved in the NHR pathway: KU70, KU80, RAD50, MRE11, XRS2, LIG4, LIF1, NEJ1 and SIR4 (van den Bosch et al., 2002, Biol. Chem. 383: 873-892 and Allen et al., 2003, Mol. Cancer Res. 1:913-920). Most preferred are one of KU70, KU80, and LIG4 and both KU70 and KU80. The decrease in expression level of the NHR component can be achieved using the methods as described herein for obtaining the deficiency of the essential gene. Since it is possible that decreasing the expression of components involved in NHR may result in adverse phenotypic effects, it is preferred that in the host cell of the vector-host system according to the invention, the increase in efficiency in homologous recombination is inducible. This can be achieved by methods known to the person skilled in the art, for example by either using an inducible process for an NHR component (e.g. by placing the NHR component behind an inducible promoter) or by using a transient disruption of the NHR component, or by placing the gene encoding the NHR component back into the genome.

The invention also relates to a method for the preparation of a library of assembled polynucleotides, which method comprises:
preparing a library of host cells as described herein; and
recovering the assembled nucleic acids from the library of host cells, thereby to prepare a library of assembled polynucleotides.

The invention also provides an assembled polynucleotide obtainable from such a library. Assembled nucleotide sequences can be isolated from the host cells using any suitable means, for example using lysis and, optionally, nucleic acid purification procedures well known to those skilled in the art or with commercially available cell lysis and DNA purification reagents and kits. The assembled polynucleotide sequences may conveniently be recovered by amplification, such as PCR. Recovery may involve only lysis, such that the assembled nucleic acid preparation is in the form of a crude cellular preparation.

Typically, such a preparation may then be used to prepare a further library of host cells—that is to say, the crude preparation may be used to introduce the assembled nucleic acids into a further set of host cells (for example host cells of a different species than the host cells used to generated the first library). The assembled polynucleotide may contain additional sequences such that homologous recombination may be carried out with a target locus in the further host cells.

However, the assembled nucleic acids may be extracted, isolated, purified or amplified from a sample (e.g., from an organism of interest or culture containing a plurality of organisms of interest, like yeast or bacteria for example).

The term “isolated” as used herein refers to nucleic acid removed from its original environment (e.g., the natural environment if it is naturally occurring, or a host cell if expressed exogenously), and thus is altered “by the hand of man” from its original environment.

An isolated nucleic acid generally is provided with fewer non-nucleic acid components (e.g., protein, lipid) than the amount of components present in a source sample. A composition comprising isolated sample nucleic acid can be substantially isolated (e.g., about 90%, 91%, 92%, 93%, 94%, 95%, 96%, 97%, 98%, 99% or greater than 99% free of non-nucleic acid components). The term “purified” as used herein refers to sample nucleic acid provided that contains fewer nucleic acid species than in the sample source from which the sample nucleic acid is derived. A composition comprising sample nucleic acid may be substantially purified (e.g., about 90%, 91%, 92%, 93%, 94%, 95%, 96%, 97%, 98%, 99% or greater than 99% free of other nucleic acid species). In this way a library of nucleic acids may be prepared.

The invention further provides a method for the preparation of a host cell having a desired property, which method comprises:
preparing a library of host cells as described herein; and
screening said library of host cells, thereby to identify a host cell with the desired property.

Also, there is provided by the invention a method for the preparation of a host cell having a desired property, which method comprises:
preparing a library of assembled polynucleotides as described herein;
transferring the library into host cells; and
screening the resulting host cells, thereby to identify a host cell with the desired property.

In these methods, after a library according to the invention has been constructed, optimized host cells comprising assembled polypeptides in the library can be selected. The initial library of host cells generated by a method of the invention may be screened. Alternatively, a nucleic acid library may be generated according to the invention and transferred into further host cells which are then screened.

Any suitable assay system can be utilized, include a system that assesses the relative, or actual amount, of, for example, a target product produced by a library species. Assay systems amenable to higher-throughput screening often is utilized to select library species that most effectively and/or efficiently produce target product. Assays may be conducted over a time course to determine library species that most quickly produce product, and identify library species that produce the most amount of product.

Libraries of host cells may be screened by culturing a host cell under conditions that optimizes yield of a target molecule. In general, conditions that may be optimized include the type and amount of carbon source, the type and amount of nitrogen source, the carbon-to-nitrogen ratio, the oxygen level, growth temperature, pH, length of the biomass production phase, length of target product accumulation phase, and time of cell harvest.

Fermentation conditions in which screening assays may be carried out can include several parameters, including without limitation, temperature, oxygen content, nutrient content (e.g., glucose content), pH, agitation level (e.g., revolutions per minute), gas flow rate (e.g., air, oxygen, nitrogen gas), redox potential, cell density (e.g., optical density), cell viability and the like. A change in fermentation conditions (e.g., switching fermentation conditions) is an alteration, modification or shift of one or more fermentation parameters. For example, one can change fermentation conditions by increasing or decreasing temperature, increasing or decreasing pH (e.g., adding or removing an acid, a base or carbon dioxide), increasing or decreasing oxygen content (e.g., introducing air, oxygen, carbon dioxide, nitrogen) and/or adding or removing a nutrient (e.g., one or more sugars or sources of sugar, biomass, vitamin and the like), or combinations of the foregoing. Fermentation conditions appropriate for specific target products and host cells are well known to those skilled in the art and the precise fermentation conditions used will depend on the specific target product and target cell.

The method of the invention may be used to identify host cells which have a desired property. Typically, this will be a property in terms of an activity in an engineered microorganism that is added or modified relative to the host microorganism (e.g., added, increased, reduced, inhibited or removed activity).

An added activity may be an activity not detectable in a host microorganism. An increased activity generally is an activity increased in a host cell selected using the invention as compared with a reference host cell (for example a host cell comprising the same pathway as comprised within the assembled polynucleotide).

An activity can be increased to any suitable level for production of a target product, including but not limited to less than about 2-fold (e.g., about 10% increase to about 99% increase; about 20%, 30%, 40%, 50%, 60%, 70%, 80%, 90% increase), 2-fold, 3-fold, 4-fold, 5-fold, 6-fold, 7-fold, 8-fold, 9-fold, of 10-fold increase, or greater than about 10-fold increase in comparison with a reference host cell.

A reduced or inhibited activity generally is an activity detectable in a host microorganism that has been reduced or inhibited in a host cell selected using the invention as compared with a reference host cell. An activity can be reduced to undetectable levels in some embodiments, or detectable levels in certain embodiments. An activity can be decreased to any suitable level for production of a target product, including but not limited to less than 2-fold (e.g., about 10% decrease to about 99% decrease; about 20%, 30%, 40%, 50%, 60%, 70%, 80%, 90% decrease), 2-fold, 3-fold, 4-fold, 5-fold, 6-fold, 7-fold, 8-fold, 9-fold, of 10-fold decrease, or greater than about 10-fold decrease.

The invention further provides a method for the preparation of a host cell having a desired property, which method comprises:
preparing a library of host cells as described herein; and
screening said library of host cells, thereby to identify a host cell with the desired property.

Also, there is provided by the invention a method for the preparation of a host cell having a desired property, which method comprises:
preparing a library of assembled polynucleotides as described herein;
transferring the library into host cells; and
screening the resulting host cells, thereby to identify a host cell with the desired property.

A library of host cells, a library of nucleic acids and a host cell having a desired property prepared according to the methods described herein are also provided by the invention. The invention further provides an assembled nucleic acid obtainable from or derived from such a host cell. Thus, the invention provides a method for the identification of an assembled nucleic acid which confers on a cell an improved property. The improved property may be the production of a desired target product.

A host cell with a desired property identified using the method of the invention may then be used for the production of a target product. The target product may be provided within cultured microbes containing target product, and cultured microbes may be supplied fresh or frozen in a liquid media or dried. Fresh or frozen microbes may be contained in appropriate moisture-proof containers that may also be temperature controlled as necessary. Target product may be provided in culture medium that is substantially cell-free. In some embodiments target product or modified target product purified from microbes is provided, and target product sometimes is provided in substantially pure form.

Amino acid or nucleotide sequences are said to be homologous when exhibiting a certain level of similarity. Two sequences being homologous indicate a common evolutionary origin. Whether two homologous sequences are closely related or more distantly related is indicated by “percent identity” or “percent similarity”, which is high or low respectively. Although disputed, to indicate “percent identity” or “percent similarity”, “level of homology” or “percent homology” are frequently used interchangeably. For the purposes of the invention, a comparison of sequences and determination of percent identity between two sequences can be accomplished using a mathematical algorithm. The skilled person will be aware of the fact that several different computer programs are available to align two sequences and determine the homology between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley).

The percent identity between two nucleic acid or amino acid sequences can be determined using the Needleman and Wunsch algorithm for the alignment of two sequences. (Needleman, S. B. and Wunsch, C. D. (1970) J. Mol. Biol. 48, 443-453). The algorithm aligns amino acid sequences as well as nucleotide sequences. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.biometrics.nl/). For protein sequences, EBLOSUM62 may be used for the substitution matrix. For nucleotide sequences, EDNAFULL may be used. Other matrices can be specified. The optional parameters used for alignment of amino acid sequences are a gap-open penalty of 10 and a gap extension penalty of 0.5. The skilled person will appreciate that all these different parameters will yield slightly different results but that the overall percentage identity of two sequences is not significantly altered when using different algorithms.

The homology or identity is the percentage of identical matches between the two full sequences over the total aligned region including any gaps or extensions. The homology or identity between the two aligned sequences may be calculated as follows: Number of corresponding positions in the alignment showing an identical amino acid or nucleic acid residue in both sequences divided by the total length of the alignment including the gaps. The identity defined as herein can be obtained from NEEDLE and is labelled in the output of the program as “IDENTITY”.

The homology or identity between the two aligned sequences may be calculated as follows: Number of corresponding positions in the alignment showing an identical amino acid or nucleic acid residue in both sequences divided by the total length of the alignment after subtraction of the total number of gaps in the alignment. The identity defined as herein can be obtained from NEEDLE by using the NOBRIEF option and is labeled in the output of the program as “longest-identity”.

Sequence identity can also be determined by hybridization assays conducted under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

The present invention is further illustrated by the following Examples:

### EXAMPLES

It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Integrating a Pathway with In Vivo Nucleic Acid Assembly

### 1.1 General Principle of In Vivo Nucleic Acid Assembly

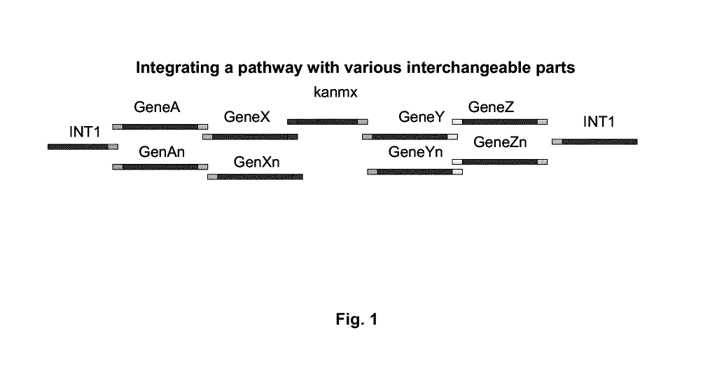

In vivo nucleic acid assembly is a technique that uses the in vivo homologous recombination system of S. cerevisiae to add diversity to pathways/metabolic routes. It is a new approach/method that is able to achieve in one step the assembly and optimization of a certain metabolic route/pathway. The technique keeps homology in the parts of a pathway that need to connect and diversity is added to the pathway where necessary. In one transformation a collection of strains is prepared having plurality of variations of the pathway. This collection is then submitted to an efficient screening method to detect the best performing strains having the best pathway variant. In this example we describe the experiments performed to demonstrate the approach. The general idea is also shown schematically in FIG. 1.

### 1.2 Preparation and Purification of PCR Fragments for Transformation

In vivo homologous recombination was used to assemble and integrate the complete test pathway into the Saccharomyces cerevisiae CEN.PK2-1C strain (MATa; ura3-52; trp1-289; leu2-3,112; his3Δ, 1; MAL2-8^{C}; SUC2). The necessary homology, in this example approximately 50 bp, on each of the PCR-fragments for recombination of the complete pathway was added to the primers used for amplification of the fragment (primer sequences are listed in Table 1, SEQ ID NOs: 1 to 14, transformed PCR products are listed as SEQ ID NOs: 15 to 24).

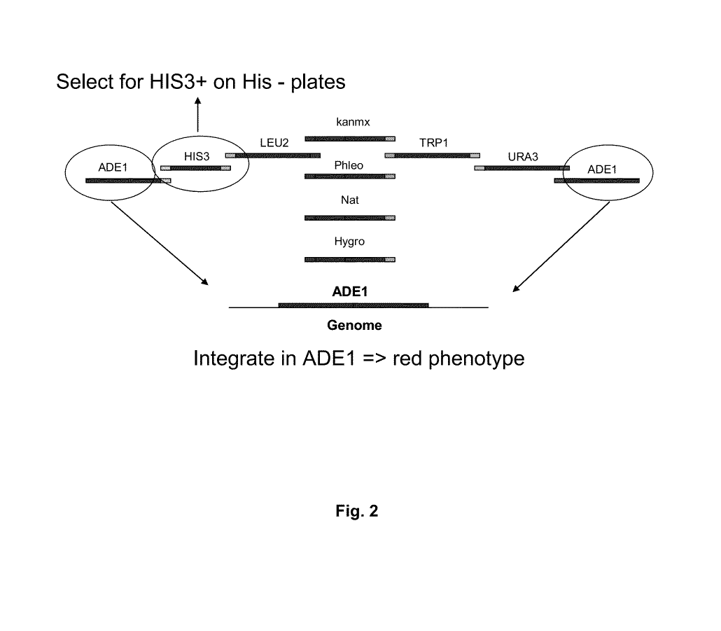

The complete integrated test pathway consists of 7 separate parts recombining into the genome. The two fragments on the edge of the pathway are the 5′ and 3′ ADE1 deletion flanks (SEQ ID NOs: 17 and 18) with overlapping homology to the test pathway. These have a functional role for integration of the pathway via a double crossover into the genome. The 5 parts in the middle are 4 expression cassettes and the marker HIS3 used for selecting transformants after transformation. From left (upstream) to right (downstream) in the pathway, the first part is a HIS3 expression cassette (used for selection), second part is a LEU2 expression cassette, third part is varied with 4 options as expression cassettes (KanMX conferring G418 resistance, Nat1 Nourseothricin resistance, Phleomycin resistance and Hgm Hygromycin resistance), fourth part is a TRP1 expression cassette and fifth part is a URA3 expression cassette. The homologous recombination event is shown in a schematic view in detail in FIG. 2.

PCR reactions were performed with Phusion polymerase (Finnzymes) according to the manual. The auxotrophic (HIS3, LEU2, TRP1 and URA3) and dominant markers (KanMX, Nat1, Phleomycin and Hygromycin), are amplified using standard plasmids containing these markers as template DNA. The 5′ and 3′ ADE1 deletion flanks were amplified using chromosomal DNA isolated from CenPK-1137d. Size of the PCR fragments was checked with standard agarose electrophoresis techniques. PCR amplified DNA fragments were purified with the PCR purification kit from Qiagen, according to the manual. DNA concentration was measured using A260/A280 on a Nanodrop ND-1000 spectrophotometer.

1.3 Transformation to S. cerevisiae

Transformation of S. cerevisiae was done as described by Gietz and Woods (2002; Transformation of the yeast by the LiAc/SS carrier DNA/PEG method. Methods in Enzymology 350: 87-96). CEN.PK113-7D (MATa URA3 HIS3 LEU2 TRP1 MAL2-8 SUC2) was transformed with 1 ug of each of the amplified and purified PCR fragments, with the exception of the fragments used in the middle with multiple options; here equal amounts of the optional fragments were used adding up to 1 ug in total. Transformation mixtures were plated on YNB-agar (67 grams per liter of Difco™ Yeast Nitrogen Base w/o Amino Acids, 20 grams per liter dextrose (Sigma), 20 grams of agar) containing 20 mg per liter adenine sulphate (Sigma), 20 mg per liter L-tryptophan (FLUKA), 100 mg per liter L-Leucin (Fluka), 50 mg per liter Uracil (Sigma) per ml. After several days of incubation at 30° C., colonies appeared on the plates, whereas the negative control (i.e., no addition of DNA in the transformation experiment) resulted in blank plates. The majority of the colonies (about 80%-90%) showed a red phenotype indicating a successful integration at the specified ADE1 locus.

### 1.4 Analysis of the Transformants

The transformation plates were used for further analysis by replica plating the transformants to plates selective for the dominant markers used in the pathway. To show the distribution of fragments in the third part of the pathway, the transformants were replica plated to G418, Nourseothricin, Phleomycin and Hygromycin selective plates. YEPD-agar (Peptone 10.0 g/l, Yeast Extract 10.0 g/l, Sodium Chloride 5.0 g/l, Agar 15.0 g/l and 2% glucose) plates were used for replica plating, the specific antibiotics were added to the plates being G418 (100 μg/ml) or Nourseothricin (100 μg/ml) or Phleomycin (15 μg/ml) or Hygromycin B (200 μg/ml). Plates were incubated at 30° C. for 2-3 days and colonies were counted and checked for their growth on one of the plates.

Results show a distribution of the resistance markers amongst the transformants, about 24% was able to grow on G418 selective plates and thus contained the KanMX marker, about 14% was able to grow on Nourseothricin selective plates and thus contained the Nat1 marker, 31% was able to grow on phleomycin selective plates and thus contained the phleomycin marker and 23% was able to grow on hygromycin selective plates and thus contained the Hygromycin resistance marker. The remaining 8% failed to grow on all plates and from that we conclude that they did not integrate the pathway correctly.

### 1.5 Chromosomal DNA Isolation

Yeast cells were grown in YEP-medium containing 2% glucose, in a rotary shaker (overnight, at 30° C. and 280 rpm). 1.5 ml of these cultures were transferred to an eppendorf tube and centrifuged for 1 minute at maximum speed. The supernatant was decanted and the pellet was resuspended in 200 μl of YCPS (0.1% SB3-14 (Sigma Aldrich, the Netherlands) in 10 mM Tris.HCl pH 7.5; 1 mM EDTA) and 1 μl RNase (20 mg/ml RNase A from bovine pancreas, Sigma, the Netherlands). The cell suspension was incubated for 10 minutes at 65° C. The suspension was centrifuged in an Eppendorf centrifuge for 1 minute at 7000 rpm. The supernatant was discarded. The pellet was carefully dissolved in 200 μl CLS (25 mM EDTA, 2% SDS) and 1 μl RNase A. After incubation at 65° C. for 10 minutes, the suspension was cooled on ice. After addition of 70 μl PPS (10M ammonium acetate) the solutions were thoroughly mixed on a Vortex mixer. After centrifugation (5 minutes in Eppendorf centrifuge at maximum speed), the supernatant was mixed with 200 μl ice-cold isopropanol. The DNA readily precipitated and was pelleted by centrifugation (5 minutes, maximum speed). The pellet was washed with 400 μl ice-cold 70% ethanol. The pellet was dried at room temperature and dissolved in 50 μl TE (10 mM Tris.HCl pH7.5, 1 mM EDTA).

TABLE 1
Primer sequences for amplification of the
fragments used in the transformation

| primer nr | | | |
|---|---|---|---|
| sequence | | Size | |
| identity | Sequence | bp | short description |
| 1496 | 5′CCGAATAATCATATGA | 20 | Forward primer for amplification of the |
| SEQ ID NO: 1 | GTCG3′ | | ADE1 5′ flank |
| 2648 | 5′ATACCTGGCAGTGAC | 75 | Reverse primer for amplification of the |
| SEQ ID NO: 2 | TCCTAGCGCTCACCAA | | ADE1 5′ flank |
| | GCTCTTAAAACGGGAAT | | |
| | TTTCGTTAATATTTCGTA | | |
| | TGTGTATTC3′ | | |
| 2649 | 5′TCGAATCATAAGCATT | 73 | Forward primer for amplification of the |
| SEQ ID NO: 3 | GCTTACAAAGAATACAC | | HIS3 expression cassette |
| | ATACGAAATATTAACGA | | |
| | AAATTCCCGTTTTAAGA | | |
| | GCTTGG3′ | | |
| 2650 | 5′TTCCCTCAAGAATTTT | 70 | Reverse primer for amplification of the |
| SEQ ID NO: 4 | ACTCTGTCAGAAACGG | | HIS3 expression cassette |
| | CCTTACGACGTAGTCG | | |
| | ATAGATCCGTCGAGTTC | | |
| | AAGAG3′ | | |
| 2651 | 5′TTCTTTTTGCTTTTTCT | 70 | Forward primer for amplification of |
| SEQ ID NO: 5 | TTTTTTTTCTCTTGAACT | | the LEU2 expression cassette |
| | CGACGGATCTATCGAC | | |
| | TACGTCGTAAGGCCGT | | |
| | TTC3′ | | |
| 2652 | 5′GAATTCGTCGACCTG | 45 | Reverse primer for amplification of |
| SEQ ID NO: 6 | CAGCGTACGAGCATAT | | the LEU2 expression cassette |
| | CGACGGTCGAGGAG3′ | | |
| 2832 | 5′AATATTAGGTATGTGG | 75 | Forward primer for amplification of the |
| SEQ ID NO: 7 | ATATACTAGAAGTTCTC | | dominant markers, phleo, Nat1, |
| | CTCGACCGTCGATATG | | hygromycin and KanMX. |
| | CTCGTACGCTGCAGGT | | |
| | CGACGAATTC3′ | | |
| 2654 | 5′GATGCTGTCTATTAAA | 75 | Reverse primer for amplification of the |
| SEQ ID NO: 8 | TGCTTCCTATATTATATA | | dominant markers, phleo, Nat1, |
| | TATAGTAATGTCGTTTT | | hygromycin and KanMX. |
| | AGGCCACTAGTGGATC | | |
| | TGATATCG3′ | | |
| 2655 | 5′AAACGACATTACTATA | 51 | Forward primer for amplification of the |
| SEQ ID NO: 9 | TATATAATATAGGAAGC | | TRP1 expression cassette |
| | ATTTAATAGACAGCATC | | |
| | G3′ | | |
| 2656 | 5′TAAAAAAAAAATGATG | 71 | Reverse primer for amplification of the |
| SEQ ID NO: 10 | AATTGAATTGAAAAGCT | | TRP1 expression cassette |
| | GTGGTATGGTGCACTC | | |
| | TTCCTGATGCGGTATTT | | |
| | TCTCC3′ | | |
| 2657 | 5′GCGGTGTGAAATACC | 70 | Forward primer for amplification of the |
| SEQ ID NO: 11 | GCACAGATGCGTAAGG | | URA3 expression cassette |
| | AGAAAATACCGCATCAG | | |
| | GAAGAGTGCACCATAC | | |
| | CACAGC3′ | | |
| 2658 | 5′ATTCAGTGAGGAGTTA | 70 | Reverse primer for amplification of the |
| SEQ ID NO: 12 | CACTGGCGACTTGTAG | | URA3 expression cassette |
| | TATATGTAAATCACGTT | | |
| | AACCGCATAGGGTAATA | | |
| | ACTG3′ | | |
| 2659 | 5′ACAAATTAGAGCTTCA | 75 | Forward primer for amplification of |
| SEQ ID NO: 13 | ATTTAATTATATCAGTTA | | ADE1 3′ flank |
| | TTACCCTATGCGGTTAA | | |
| | CGTGATTTACATATACT | | |
| | ACAAGTC3′ | | |
| 1499 | 5′TATTGACTGCGCTCTA | 24 | Reverse primer for amplification of |
| SEQ ID NO: 14 | TAAATGTC3′ | | ADE1 3′ flank |

### Example 2

### Using In Vivo Nucleic Acid Assembly to Build and Find Improved Itaconic Acid Producing Yeast Strains

### 2.1 Preparation and Purification of PCR Fragments for Transformation

In vivo homologous recombination was used to assemble and integrate itaconic acid pathway variants into Saccharomyces cerevisiae CEN.PK113-7D (MATa URA3 HIS3 LEU2 TRP1 MAL2-8 SUC2) strain with the method as described in example 1. In the current design, a itaconic acid pathway is formed by 9 separate DNA fragments recombining and integrated into the genome. In this example, each part is prepared by PCR amplification and the necessary homologous sequences between each of the PCR-fragments for recombination of the complete pathway are unique 50-bp sequences flanking each fragment. The first and last fragments of the recombined itaconic pathway construct are integration flanks providing the homology to the genomic locus where the pathway is designed to integrate into the genome. The integration flanks have 50-bp homology inward to the first fragment of the respective connecting pathway fragments; the outward sequence is the homology for the integration flank into the genome. The 7 fragments in the middle are expression cassettes (promoter, open reading frame, terminator), 6 of them are putative functional elements in the itaconic acid pathway variants as designed, and one of them is the KanMX marker cassette for G418 resistance. The primers to amplify the designed cassettes and the integration flanks are listed as SEQ ID NOs: 25 to 42. The sequences of the expression cassettes (promoter, open reading frame and terminator) used to form the pathway variants are listed as SEQ ID NOs: 43 to 54.

The functional role of the integration flanks on the edge of the pathway is improving the efficiency of integration of the pathway via a double cross over into the genome. The 7 parts in the middle are described hereafter from left (upstream) to right (downstream) in the pathway. First part, after the left integration flank, is the cassette 117 containing a S. cerevisiae ACT1 promoter expressing an itaconic acid transporter Q0C8L2 and S. cerevisiae ADH1 terminator. Second part is the marker cassette KanMX used for selecting the transformants on plates containing G418. Third part has 2 options to integrate, the cassette 120, containing the S. cerevisiae TDH3 promoter expressing the mCAD3 ORF (open reading frame) with S. cerevisiae TDH1 terminator or cassette 121 containing the same promoter and terminator but expressing mCAD2. For the fourth part in the pathway there are 4 options to integrate into the genome, cassette 133 (S. cerevisiae FBA1 promoter expressing the ACO1 ORF with S. cerevisiae GPM1 terminator), cassette 135 (S. cerevisiae FBA1 promoter expressing the ACO3 ORF with S. cerevisiae GPM1 terminator), cassette 144 (S. cerevisiae PRE3 promoter expressing ACO1 with S. cerevisiae GPM1 terminator) or cassette 146 (S. cerevisiae PRE3 promoter expressing ACO3 with S. cerevisiae GPM1 terminator). These four options create variation in the promoter strength, FBA1 promoter being stronger and PRE3 being weaker and variation in the expressed gene, ACO1 or ACO3. Fifth part is cassette 136 (S. cerevisiae PGK1 promoter expressing the ORF PYC2 with S. cerevisiae TPI1 terminator). For the sixth part, there are 2 options, cassette 137 (S. cerevisiae TEF1 promoter expressing S. cerevisiae ORF CIT1 with S. cerevisiae PDC1 terminator) or cassette 139 (S. cerevisiae TEF1 promoter expressing an E. coli variant of CIT1 with S. cerevisiae PDC1 terminator). Seventh part is cassette 140 (S. cerevisiae ENO2 promoter expressing ACDH67 with S. cerevisiae TAL1 terminator).

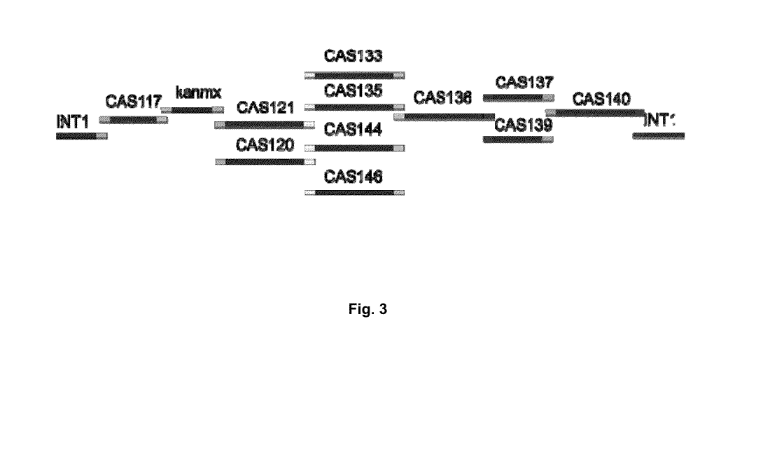

In total, 2×4×2=16 different pathway variants can theoretically be formed from this library of cassettes. The homologous recombination event might lead to 16 different pathway variants and is shown in a schematic view in FIG. 3.

PCR reactions to amplify DNA fragments were performed with Phusion polymerase (Finnzymes) according to the manual. The expression cassettes and dominant marker KanMX are amplified using standard plasmids containing the fragments as template DNA. The 5′ and 3′ INT1 deletion flanks were amplified by PCR amplification using CEN.PK113-7D genomic DNA as template. Size of the PCR fragments was checked with standard agarose electrophoresis techniques. PCR amplified DNA fragments were purified with the NucleoMag® 96 PCR magnetic beads kit of Macherey-Nagel, according to the manual. DNA concentrations were measured using the Trinean DropSense® 96 of GC biotech.

2.2 Transformation to S. cerevisiae

Transformation of S. cerevisiae was according Gietz and Woods (2002; Transformation of the yeast by the LiAc/SS carrier DNA/PEG method. Methods in Enzymology 350: 87-96). CEN.PK113-7D (MATa URA3 HIS3 LEU2 TRP1 MAL2-8 SUC2) was transformed with 400 ng of each of the amplified and purified PCR fragments, with the exception of the fragments used with multiple options; for the library fragments, equal amounts of the optional fragments were used adding up to 400 ng in total. Transformation mixtures were plated on YEPhD-agar (BBL Phytone peptone 20.0 g/l, Yeast Extract 10.0 g/l, Sodium Chloride 5.0 g/l, Agar 15.0 g/l and 2% glucose) containing G418 (400 μg/ml). After 3 days of incubation at 30° C., colonies appeared on the plates, whereas the negative control (i.e., no addition of DNA in the transformation experiment) resulted in blank plates.

### 2.3 MTP Growth Experiments for Itaconic Acid Production

Single colonies were picked and transferred to a MTP agar well containing 200 μl YEPhD-agar containing 400 μg/ml G418. After 3 days of incubation of the plate at 30° C., good grown colonies were inoculated by transferring some colony material with a pin tool in a MTP plate with standard lid containing in each well 200 μL Verduyn medium (Verduyn et al., Yeast 8:501-517, 1992, where the (NH4)2SO4 was replaced with 2 g/l Urea) with 4% galactose. The MTP was incubated in a MTP shaker (INFORS HT Multitron) at 30° C., 550 rpm and 80% humidity for 72 hours. After this pre-culture phase a production phase was started by transferring 80 μl of the broth to 2.5 ml Verduyn media (again with the urea replacing (NH4)2SO4) containing 8% galactose. After 3 days growth in a shaker at 550 rpm, 30° C. and 80% humidity the plates were centrifuged for 10 minutes at 2750 rpm in a Heraeus Multifuge 4. Supernatant was transferred to MTP plates and itaconic acid levels in the supernatant were measured using a LC-MS method.

### 2.4 Itaconic Acid Analysis Using LC-MS

UPLC-MS/MS analysis method was used for the determination of itaconic acid. A Waters HSS T3 column 1.7 μm, 100 mm*2.1 mm was used for the separation of itaconic acid from other compounds with gradient elution. Eluens A consists of LC/MS grade water, containing 0.1% formic acid, and eluens B consists of acetonitrile, containing 0.1% formic acid. The flow-rate was 0.35 ml/min and the column temperature was kept constant at 40° C. The gradient started at 95% A, and was increased linear to 30% B in 10 minutes, kept at 30% B for 2 minutes, then immediately to 95% A and stabilized for 5 minutes. The injection volume used was 2 ul. A Waters Xevo API was used in electrospray (ESI) in negative ionization mode, using multiple reaction monitoring (MRM). The ion source temperature was kept at 130° C., whereas the desolvation temperature is 350° C., at a flow-rate of 500 L/hr.

For itaconic acid, the deprotonated molecule was fragmented with 10 eV, resulting in specific fragments from losses of H2O and CO2. The standard of reference compounds spiked in blank fermentation broth were analyzed to confirm retention time, calculate a response factor for the respective ions, and was used to calculate the concentrations in fermentation samples. All samples were diluted appropriately (5-100 fold) in eluens A to overcome ion suppression and matrix effects during LC-MS analysis. Accurate mass analysis of itaconic acid to confirm the elemental composition of the compound analyzed accurate mass analyses was performed with the same chromatographic system as described above, coupled to a LTQ orbitrap (ThermoFisher). Mass calibration was performed in constant infusion mode, using a NaTFA mixture (ref), in such a way that during the experimental set-up the accurate mass analyzed could be fitted within 2 ppm from the theoretical mass, of the compound analyzed.

### 2.5 Results of the Itaconic Acid Fermentation Experiment

Table 2 shows the itaconic acid production levels of the strains that had grown well on the MTP plate with G418. The itaconic acid production levels clearly show significant variation. The complete set was used for further characterization with PCR; results are also shown in Table 2. The PCR reactions were used to determine which of the cassettes integrated in the strains. This data was applied to learn if there is a correlation between the production levels and introduced variants of cassettes within the pathway for the fragments where variation was introduced. Paragraph 1.6 and 1.7 describe the experimental steps of chromosomal DNA isolation and PCR.

2.6 Chromosomal DNA Isolation with YeaStar Genomic DNA Kit™ (ZYMO Research)

Inoculation of the strains in a 24-well plate containing 1 ml YephD (2% glucose) and ON incubation at 30° C., 550 rpm and 80% humidity in a shaker. OD660 was measured with a biochrom Ultrospec 2000 spectrophotometer to obtain the right amount of cells (1-5×10⁷ cells) as described in the manual of the kit. The isolation proceeded as described in Protocol II in the manual of the YeaStar Genomic DNA Kit™. After isolation, the DNA concentration was checked with a Nanodrop ND-1000 (Thermo Scientific), concentrations were low, in the order of 10 ng/μl, but suitable enough for PCR purposes.

### 2.7 PCR and Genetic Characterization of the Itaconic Acid Cassette Variation in Itaconic Acid Producing Strains

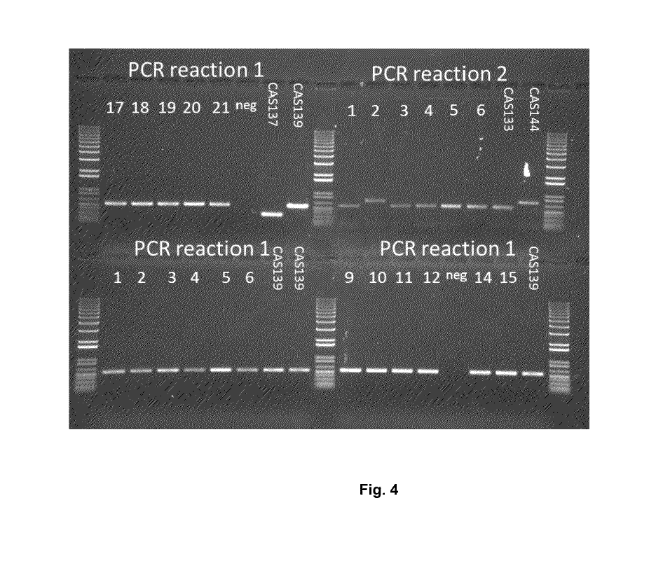

All PCR reactions were performed with Phusion polymerase and setup according to the manual. Approximately 20 to 50 ηg chromosomal DNA was used in each of the PCR reactions as template. A primer concentration of 0.2 μM was used in the reaction for each individual primer. Chromosomal DNA isolated from the CEN.PK113-7D without the specific cassettes was used as a negative control for each reaction, mentioned as “neg” in Table 2. The original cassettes or strains containing the cassettes were used as positive controls for the reactions, mentioned as “pos” in Table 2. First series of PCR reactions for the strains listed in Table 2 were carried out with primers listed as “SEQ ID NO: 57”, “SEQ ID NO: 58” and “SEQ ID NO: 59”. These PCR reactions were used to determine the presence of cassette 139 or cassette 137 in one PCR reaction. The primer SEQ ID NO: 57 is specific for cassette 137 and forms with primer “SEQ ID NO: 58” a PCR product of 333 bp. The primer with SEQ ID NO: 58 is specific for cassette 139 and forms with primer “SEQ ID NO: 59” a PCR product of 548 bp. The PCR reactions were set up with the combination of the primes and analysis of the PCR on a standard 0.8% agarose gel showed that only cassette 139 was found in the set of strains. FIG. 4 shows the results from the analysis of the PCR reactions on gel. This PCR reaction is named PCR reaction 1 and numbers for each lane are used to identify each strain and relate back to the numbers in Table 2 summarizing the outcome of all PCR's and itaconic acid production

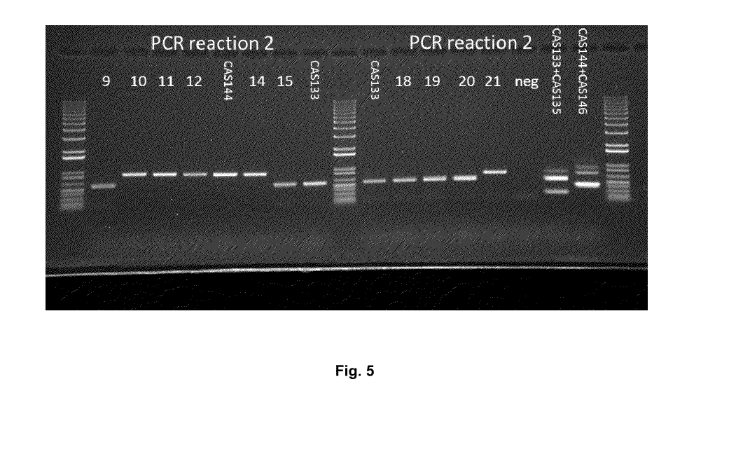

Second series of PCR reactions for each strain listed in Table 2 were done with primers listed as “SEQ ID NO: 60”, “SEQ ID NO: 61”, “SEQ ID NO: 62” and “SEQ ID NO: 63. These PCR reactions were used to determine the presence of cassette 133, cassette 135, cassette 144 or cassette 146 in one PCR reaction. Primer combination SEQ ID NO: 60 with SEQ ID NO: 63 is specific for cassette 133 and forms a PCR product of 577 bp. Primer combination SEQ ID NO: 60 with SEQ ID NO: 61 is specific for cassette 135 and forms a PCR product of 259 bp. Primer combination SEQ ID NO: 61 with SEQ ID NO: 62 is specific for cassette 146 and forms a PCR product of 430 bp. Primer combination SEQ ID NO: 61 with SEQ ID NO: 63 is specific for cassette 144 and forms a PCR product of 748 bp. When the combination of all primes was used in the reaction the resulting PCR products analyzed on a standard 0.8% agarose gel showed that cassette 133 and cassette 144 were found in the set of strains. FIGS. 4 and 5 show the results from the analysis of the PCR reactions on gel. This PCR reaction is named “PCR reaction 2” and numbers for each lane are used to identify each strain and relate back to the numbers in table n summarizing the outcome of all PCR's and itaconic acid production

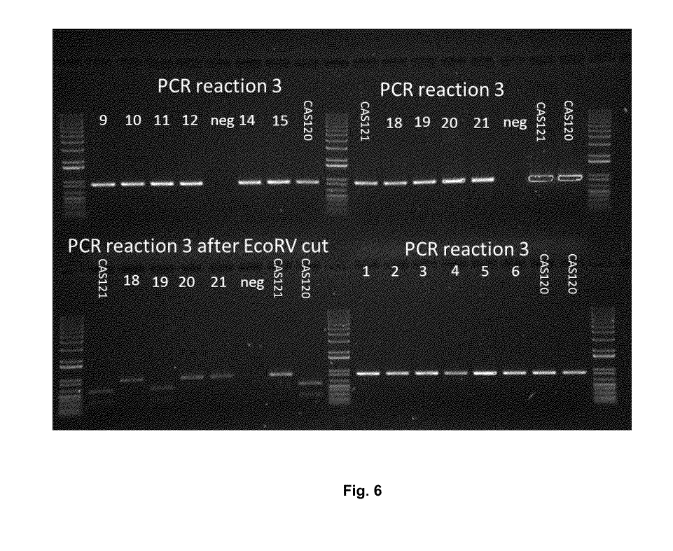

Final series of PCR reactions for the strains listed in the result table was done with primers listed as SEQ ID NO: 55 and SEQ ID NO: 56. These PCR reactions were used to determine the presence of cassette 120 or cassette 121 in the PCR reaction. The primers are specific for both cassettes and form a PCR product of 881 bp. When the combination of primes was used in the reaction the resulting PCR products analyzed on a standard 0.8% agarose gel showed that all strains contained either cassette 120 or cassette 121. FIG. 6 shows the results from the analysis of the PCR reactions on gel. This PCR reaction is named “PCR reaction 3” and numbers for each lane are used to identify each strain and relate back to the numbers in Table 2 summarizing the outcome of all PCR's and itaconic acid production.

In order to determine which cassette was integrated in the strains the restriction enzyme EcoRV was used to cut the obtained PCR fragments. The sequence of cassette 121 contains an EcoRV site whereas the cassette 120 does not contain an EcoRV recognition site. Cutting the PCR product of cassette 121 with EcoRV results in a fragment of size 584 bp and a fragment of size 297 bp, PCR product of cassette 120 remains the same size when incubated with EcoRV.

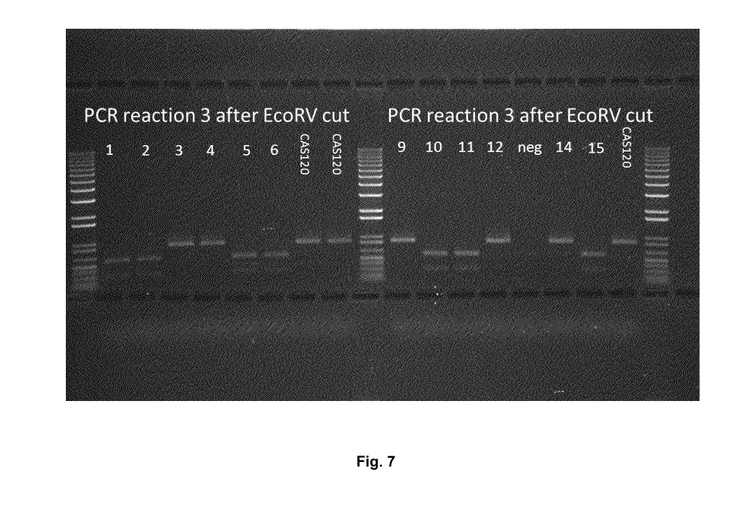

From the PCR reactions containing the PCR products of each strain, 5 μl was combined with 2 μl buffer React2 (Invitrogen), 12 μl milliQ and 1 μl EcoRV (1000 Units/μl from Invitrogen). The RE digestion was incubated at 37° C. for 2 hours and subsequently analyzed on a standard 0.8% agarose gel showing that the strains contained either cassette 120 or cassette 121 as shown in Table 2. FIGS. 6 and 7 show the results of the PCR reactions cut with EcoRV analyzed on gel. This is named “PCR reaction 3 after EcoRV cut” and numbers for each lane are used to identify each strain and controls and relate back to the numbers in Table 2 summarizing the outcome of all PCR's, further genetic analysis with the EcoRV cut and itaconic acid production.

TABLE 2
Overview of itaconic acid producing strains and characterization
of introduced pathway fragments. A clear positive correlation
is observed for mCAD2and high itaconic acid production.
Further details are given in the text.

| strain nr | | | PCR | Itaconic |
|---|---|---|---|---|
| correlating | PCR | PCR | reaction | acid |
| with gel | reaction 1 | reaction 2 | 3 + EcoRV | (mg/l) |
| 10 | CAS139 | CAS144 | mCAD2(CAS121) | 779 |
| | (E. coli CIT1) | | | |
| 11 | CAS139 | CAS144 | mCAD2(CAS121) | 731 |
| | (E. coli CIT1) | | | |
| 15 | CAS139 | CAS133 | mCAD2(CAS121) | 729 |
| | (E. coli CIT1) | | | |
| 1 | CAS139 | CAS133 | mCAD2(CAS121) | 726 |
| | (E. coli CIT1) | | | |
| 5 | CAS139 | CAS133 | mCAD2(CAS121) | 690 |
| | (E. coli CIT1) | | | |
| 6 | CAS139 | CAS133 | mCAD2(CAS121) | 672 |
| | (E. coli CIT1) | | | |
| 3 | CAS139 | CAS133 | mCAD3 (CAS120) | 645 |
| | (E. coli CIT1) | | | |
| 18 | CAS139 | CAS133 | mCAD3 (CAS120) | 640 |
| | (E. coli CIT1) | | | |
| 19 | CAS139 | CAS133 | mCAD2(CAS121) | 639 |
| | (E. coli CIT1) | | | |
| 14 | CAS139 | CAS144 | mCAD3 (CAS120) | 638 |
| | (E. coli CIT1) | | | |
| 12 | CAS139 | CAS144 | mCAD3 (CAS120) | 636 |
| | (E. coli CIT1) | | | |
| 20 | CAS139 | CAS133 | mCAD3 (CAS120) | 636 |
| | (E. coli CIT1) | | | |
| 9 | CAS139 | CAS133 | mCAD3 (CAS120) | 631 |
| | (E. coli CIT1) | | | |
| 4 | CAS139 | CAS133 | mCAD3 (CAS120) | 585 |
| | (E. coli CIT1) | | | |
| 21 | CAS139 | CAS144 | mCAD3 (CAS120) | 549 |
| | (E. coli CIT1) | | | |
| 2 | CAS139 | CAS144 | mCAD2(CAS121) | 536 |
| | (E. coli CIT1) | | | |

Genetic characterization of the introduced itaconic acid of 16 well producing strains is provided in Table 2. Amongst this set we find strains that contain CAS139, CAS143, CAS144, CAS121 and CAS120. Cassettes CAS137, CAS 135 and CAS146 were not detected.

A correlation exists between itaconic acid production and the presence of either cassette 120 or cassette 121. Strains with cassette 121 (mCAD2) clearly show significant higher itaconic acid production and are dominant in the top 6 of the itaconic acid producing strains tested. Preference for either cassette 133 and cassette 144 cannot be separated based on the observed itaconic acid production in this experiment. CAS 135 and CAS146 are not observed, indicating that the promoters associated with the respective genes are either too weak or too strong to lead to a reasonable production of itaconic acid, or lead to not-viable or not well-growing cells. Cassette 137 was not observed. Overall, with this example we have shown the use of the “in vivo nucleic acid assembly” method to create combinatorial diversity in strains with a single transformation using mixes of fragments resulting in a set of itaconic acid producing strains, with varying production levels. Genomic characterization of the introduced pathway fragments, shows that the method can be applied to select for alternative pathways genes and/or cassettes with variation in operating sequences, like for examples promoter sequences varying in transcriptional strength. This method can be applied for pathway tuning and selection of improved strains, and the subsequent deprival of contributing sequences.

1. A method for the preparation of a library of host cells, a plurality of which comprise an assembled polynucleotide at a target locus, which method comprises:
(a) providing a plurality of polynucleotides comprising two or more polynucleotide subgroups, wherein:
(i) a plurality of polynucleotides in each polynucleotide subgroup comprises sequence encoding a peptide or polypeptide and/or a regulatory sequence;
(ii) a plurality of peptides or polypeptides encoded by, or a plurality of regulatory sequences comprised within, each polynucleotide subgroup share an activity and/or function;
(iii) at least one polynucleotide subgroup comprises at least two non-identical polynucleotide species;
(iv) a plurality of polynucleotides of each polynucleotide subgroup comprises sequence enabling homologous recombination with a plurality of polynucleotides from one or more other polynucleotide subgroups; and
(v) a plurality of polynucleotides in two polynucleotide subgroups comprise a nucleotide sequence enabling homologous recombination with a target locus in host cells; and
(b) assembling the plurality of polynucleotides at the target locus by homologous recombination in vivo in host cells,
thereby to generate a library of host cells, a plurality of which comprise an assembled polynucleotide at the target locus.

2. A method according to claim 1, wherein there are at least about four polynucleotide subgroups.

3. A method according to claim 1, wherein there are about 20 or fewer polynucleotide subgroups.

4. A method according to claim 1, wherein in (v), a plurality of polynucleotides in one of the two polynucleotide subgroups is capable of homologous recombination with a 5′ sequence of the target locus and a plurality of polynucleotides in the other of the two polynucleotide subgroups is capable of homologous recombination with a 3′ sequence of the target locus.

5. A method according to claim 1, wherein a plurality of polynucleotides in at least one polynucleotide subgroup comprise sequence encoding a marker gene, with or without at least one regulatory sequence.

6. A method according to claim 1, wherein at least two polynucleotides within at least two polynucleotide subgroups are non-identical.

7. A method according to claim 1, wherein at least two polynucleotides within all of polynucleotide subgroups, other than the two polynucleotide subgroups comprising sequence enabling homologous recombination with a target locus and any polynucleotide subgroup comprising sequence encoding a marker gene, are non-identical.

8. A method according to claim 1, wherein at least about 50% of host cells in the library harbour at least one assembled polynucleotide at one or more target loci.

9. A method according to claim 1, wherein at least about 70% of the host cells in the library harbour at least one assembled polynucleotide which comprises one polynucleotide from each polynucleotide subgroup.

10. A method according to claim 1, wherein the library of host cells includes at least about 1000 different assembled polynucleotides.

11. A method according to claim 1, wherein at least one assembled polynucleotide comprises each member of a biological pathway.

12. A method according to claim 11, wherein the biological pathway enables production of a compound of interest in the host cell.

13. A method according to claim 12, wherein the compound of interest is a primary metabolite, a secondary metabolite, a polypeptide and/or a mixture of polypeptides.

14. A method according to claim 1, wherein at least one polynucleotide subgroup encodes variants of a polypeptide and/or comprises variants of a regulatory sequence.

15. A method according to claim 14, wherein the variants comprise members of a gene cluster.

16. A method according to claim 14, wherein the variants are allelic or species variants of a polypeptide or regulatory sequence.

17. A method according claim 14, wherein the variants are artificial variants.

18. A method according to claim 14, wherein the variants all share at least about 50% sequence identity with each other.

19. A method according to claim 1, wherein a plurality of polynucleotides in a subgroup encoding a polypeptide is operably linked with a promoter.

20. A method according to claim 19, wherein each of the plurality of polynucleotides in a subgroup is operably linked to one promoter and wherein the subgroup comprises at least two different promoters.

21. A method according to claim 1, wherein each of the plurality of polynucleotides comprising two or more polynucleotide subgroups is from about 50 bp to about 10 kbp in length.

22. A method according to claim 1, wherein the sequences enabling homologous recombination are from 20 bp to 5 kb in length.

23. A method according to claim 1, wherein the target locus is a locus within the genome of the host cell.

24. A method of claim 1, wherein the target locus is an extra-chromosomal target locus.

25. A method according to claim 24, wherein the extra-chromosomal target locus is a plasmid or an artificial chromosome.

26. A method according to claim 1, wherein the host cells are prokaryotic or eukaryotic cells.

27. A method according to claim 26, wherein the prokaryotic cells are bacterial cells.

28. A method according to claim 26, wherein the eukaryotic host cells are fungal cells, yeast cells, mammalian cells or insect cells.

29. A method according to claim 28, wherein the yeast cells are S. cerevisiae cells.

30. A method for the preparation of a library of assembled polynucleotides, which method comprises:
preparing a library of host cells according to claim 1; and
recovering the assembled polynucleotides from the library of host cells,
thereby to prepare a library of assembled polynucleotides.

31. A method for identification of a host cell having a desired property, which method comprises:
preparing a library of host cells according to claim 1; and
screening said library of host cells,
thereby to identify a host cell with the desired property.

32. A method for the preparation of a host cell having a desired property, which method comprises:
preparing a library of assembled polynucleotides according to claim 30;
transferring the library into host cells; and
screening the resulting host cells,
thereby to identify a host cell with the desired property.

33. A library of host cells prepared according to the method of claim 1.

34. A library of assembled polynucleotides prepared according to the method of claim 30.

35. A host cell having a desired property prepared according to the method of claim 31.

36. An assembled nucleic acid derived from a library according to claim 34.

37. A method for expression screening of filamentous fungal transformants, comprising:
(a) isolating single colony transformants of a library of yeast host cells prepared by a method according to claim 1;
(b) preparing DNA from the single colony of yeast transformants;
(c) introducing a sample of the preparations of (b) into separate suspensions of protoplasts of a filamentous fungus to obtain transformants thereof, wherein transformants contain one or more copies of an individual polynucleotide from the library of yeast host cells;
(d) growing individual filamentous fungal transformants of (c) on selective growth medium, thereby permitting growth of the filamentous fungal transformants, while suppressing growth of untransformed filamentous fungi; and
(e) measuring activity or a property of each polypeptide encoded by the individual polynucleotides.


SEQUENCE LISTING (63 sequences)

SEQ ID NO: 1 - DNA, 20 bp, Artificial Sequence
  Forward primer for amplification of the ADE1 5′ flank
ccgaataatcatatgagtcg

SEQ ID NO: 2 - DNA, 75 bp, Artificial Sequence
  Reverse primer for amplification of the ADE1 5′ flank
atacctggcagtgactcctagcgctcaccaagctcttaaaacgggaattttcgttaatatttcgtatgtgtattc

SEQ ID NO: 3 - DNA, 73 bp, Artificial Sequence
  Forward primer for amplification of the HIS3 expression cassette
tcgaatcataagcattgcttacaaagaatacacatacgaaatattaacgaaaattcccgttttaagagcttgg

SEQ ID NO: 4 - DNA, 70 bp, Artificial Sequence
  Reverse primer for amplification of the HIS3 expression cassette
ttccctcaagaattttactctgtcagaaacggccttacgacgtagtcgatagatccgtcgagttcaagag

SEQ ID NO: 5 - DNA, 70 bp, Artificial Sequence
  Forward primer for amplification  of the LEU2 expression cassette
ttctttttgctttttctttttttttctcttgaactcgacggatctatcgactacgtcgtaaggccgtttc

SEQ ID NO: 6 - DNA, 45 bp, Artificial Sequence
  Reverse primer for amplification  of the LEU2 expression cassette
gaattcgtcgacctgcagcgtacgagcatatcgacggtcgaggag

SEQ ID NO: 7 - DNA, 75 bp, Artificial Sequence
  Forward primer for amplification of the dominant markers, phleo, Nat1, hygromycin and KanMX
aatattaggtatgtggatatactagaagttctcctcgaccgtcgatatgctcgtacgctgcaggtcgacgaattc

SEQ ID NO: 8 - DNA, 75 bp, Artificial Sequence
  Reverse primer for amplification of the dominant markers, phleo, Nat1, hygromycin and KanMX
gatgctgtctattaaatgcttcctatattatatatatagtaatgtcgttttaggccactagtggatctgatatcg

SEQ ID NO: 9 - DNA, 51 bp, Artificial Sequence
  Forward primer for amplification of the TRP1 expression cassette
aaacgacattactatatatataatataggaagcatttaatagacagcatcg

SEQ ID NO: 10 - DNA, 71 bp, Artificial Sequence
  Reverse primer for amplification of the TRP1 expression cassette
taaaaaaaaaatgatgaattgaattgaaaagctgtggtatggtgcactcttcctgatgcggtattttctcc

SEQ ID NO: 11 - DNA, 70 bp, Artificial Sequence
  Forward primer for amplification of the URA3 expression cassette
gcggtgtgaaataccgcacagatgcgtaaggagaaaataccgcatcaggaagagtgcaccataccacagc

SEQ ID NO: 12 - DNA, 70 bp, Artificial Sequence
  Reverse primer for amplification of the URA3 expression cassette
attcagtgaggagttacactggcgacttgtagtatatgtaaatcacgttaaccgcatagggtaataactg

SEQ ID NO: 13 - DNA, 75 bp, Artificial Sequence
  Forward primer for amplification of ADE1 3′ flank
acaaattagagcttcaatttaattatatcagttattaccctatgcggttaacgtgatttacatatactacaagtc

SEQ ID NO: 14 - DNA, 24 bp, Artificial Sequence
  Reverse primer for amplification of ADE1 3′ flank
tattgactgcgctctataaatgtc

SEQ ID NO: 15 - DNA, 1032 bp, Artificial Sequence
  PCR fragment “5′ ADE1 flank” with homology to part 1 (HIS3) in the test pathway
ccgaataatcatatgagtcgggcaattccgaagaattttctttgtaatgtcgctgatcccttttactgttgctatgcatatgtttttccacgttctcctcctctaactctttgtcatcatctctatttcgcagaacatcatggcccttttctgccgcattactcagtatattaagtttcgaattgaagggcgaactcttattcgaagtcggagtcaccacaacacttccgcccatactctccgaatcctcgtttcctaaagtaagtttacttccacttgtaggcctattattaatgatatctgaataatcctctattagggttggatcattcagtagcgcgtgcgattgaaaggagtccatgcccgacgtcgacgtgattagcgaaggcgcgtaaccattgtcatgtctagcagctatagaactaacctccttgacaccacttgcggaagtctcatcaacatgctcttccttattactcattctcttaccaagcagagaatgttatctaaaaactacgtgtatttcacctctttctcgacttgaacacgtccaactccttaagtactaccacagccaggaaagaatggatccagttctacacgatagcaaagcagaaaacacaaccagcgtacccctgtagaagcttctttgtttacagcacttgatccatgtagccatactcgaaatttcaactcatctgaaacttttcctgaaggttgaaaaagaatgccataagggtcacccgaagcttattcacgagtcagtctgactcttgcgagagatgaggatgtaataatactaatctcgaagatgccatctaatacatatagacatacatatatatatatatacattctatatattcttacccagattctttgaggtaagacggttgggttttatcttttgcagttggtactattaagaacaatcgaatcataagcattgcttacaaagaatacacatacgaaatattaacgaaaattcccgttttaagagcttggtgagcgctaggagtcactgccaggtat

SEQ ID NO: 16 - DNA, 1050 bp, Artificial Sequence
  PCR fragment “3′ ADE1 flank” with homology to part 5 (URA3) in the test pathway
acaaattagagcttcaatttaattatatcagttattaccctatgcggttaacgtgatttacatatactacaagtcgccagtgtaactcctcactgaatatgattcatacatacccgtatgtattaatgtataaatgttctcagagcaaattttatcgatatcttgtttgccagtggtatgcaggtttggcaaattttttaccataatatccgtttatagattctggaaccttaccaactttcttaccgctaattacttccctggctcgctcctccactgcctgggtaaattgttccttcaactgactcagttctctttcgtattcaatagcttgcttctcgaggattttttcaatgtttgtcagctcattttcatagtccagtaacttcctttcaaatctctctaattgcaacgactttcttgcagttcgtatctgaatatcttgcagtaattcaaaagtggaaggcctggttcttaagttcacatctatcattgaatgtattatggcattaagccctctagagtaatactcagggacggtgtcacatttcccgtttttaatcttagtttgtagctcgagataattttttgcctgaaatggggggtgcaacgaacacatctcaaaaataacacaacctagtgaccagatgtcggatagtggggagtatggttggtccatcaacacttcaggcgacatgtaatatggtgtaccgacgtatgttgtggcaaattgaatactagtttccagagatttggctaacccaaaatcacctaactttaccacaacttgactatagtccatagggctcccccttttccctgaattcactctatggtctctgtaataattactattcacttcctcgtgaccgtctacttgttcattaatattgtaatcgctatcatcatagcttaagaatatatttcctggtttcagatcacgatggataacgatgtttttgccttttaccggtggtttcatccggtcatatattgtggtcaaagttggcaattcaacaccataatgacatttatagagcgcagtcaata

SEQ ID NO: 17 - DNA, 1286 bp, Artificial Sequence
  HIS3 expression cassette
tcgaatcataagcattgcttacaaagaatacacatacgaaatattaacgaaaattcccgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttcccctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgacggatctatcgactacgtcgtaaggccgtttctgacagagtaaaattcttgagggaa

SEQ ID NO: 18 - DNA, 2311 bp, Artificial Sequence
  LEU2 expression cassette
ttctttttgctttttctttttttttctcttgaactcgacggatctatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtgggacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacataggggcagacattagaatggtatatccttgaaatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgctcgtacgctgcaggtcgacgaattc

SEQ ID NO: 19 - DNA, 1672 bp, Artificial Sequence
  Kanmx expression cassette (G418 resistance)
aatattaggtatgtggatatactagaagttctcctcgaccgtcgatatgctcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgatatcagatccactagtggcctaaaacgacattactatatatataatataggaagcatttaatagacagcatc

SEQ ID NO: 20 - DNA, 1249 bp, Artificial Sequence
  ble expression cassette (phleomycin resistance)
aatattaggtatgtggatatactagaagttctcctcgaccgtcgatatgctcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtatgaccgaccaagcgacgcccaacctgccatcacgagatttcgattccacggccgccttctatgaaaggttgggcttcggaatcgttttccgggacgccggctggatgatcctccagcgcggggatctcaagctggagttcttcgcccaccccgggctcgatcccctcgcgagttggttcagctgctgcctgaggctggacgacctcgcggagttctaccggcagtgcaaatccgtcggcatccaggaaaccagcagcggctatccgcgcatccatgcccccgaactgcaggagtggggaggcacgatggccgctttggtcgacccggacgggacgctcctgcgcctgatacagaacgaattgcttgcaggcatctcatgatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgatatcagatccactagtggcctaaaacgacattactatatatataatataggaagcatttaatagacagcatc

SEQ ID NO: 21 - DNA, 1435 bp, Artificial Sequence
  Nat1 expression cassette (Nourseothricin resistance)
aatattaggtatgtggatatactagaagttctcctcgaccgtcgatatgctcgtacgctgcaggtcgacgaattctaccgttcgtataatgtatgctatacgaagttatagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgacggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgatatcagatccactagtggcctaaaacgacattactatatatataatataggaagcatttaatagacagcatc

SEQ ID NO: 22 - DNA, 1891 bp, Artificial Sequence
  Hygromycin resistance expression cassette
aatattaggtatgtggatatactagaagttctcctcgaccgtcgatatgctcgtacgctgcaggtcgacgaattctaccgttcgtataatgtatgctatacgaagttatagatctgtttagcttgccttgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgatatcagatccactagtggcctaaaacgacattactatatatataatataggaagcatttaatagacagcatc

SEQ ID NO: 23 - DNA, 1106 bp, Artificial Sequence
  TRP1 expression cassette
aaacgacattactatatatataatataggaagcatttaatagacagcatcgtaatatatgtgtactttgcagttatgacgccagatggcagtagtggaagatattctttattgaaaaatagcttgtcaccttacgtacaatcttgatccggagcttttctttttttgccgattaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctatgcggtgtgaaataccgcacagatgcgtaaggagaaaataccgcatcaggaagagtgcaccataccacagcttttcaattcaattcatcatttttttttta

SEQ ID NO: 24 - DNA, 1233 bp, Artificial Sequence
  URA3 expression cassette
gcggtgtgaaataccgcacagatgcgtaaggagaaaataccgcatcaggaagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggttaacgtgatttacatatactacaagtcgccagtgtaactcctcactgaat

SEQ ID NO: 25 - DNA, 19 bp, Artificial Sequence
  forward primer for amplification of 5 integration flank INT1
cggcattattgtgtatggc

SEQ ID NO: 26 - DNA, 73 bp, Artificial Sequence
  reverse primer for amplification of 5 integration flank INT1
aaacgcctgtgggtgtggtactggatatgcaaagcgattggaagtcgcttagggtttcaaagatccatacttc

SEQ ID NO: 27 - DNA, 22 bp, Artificial Sequence
  forward primer for amplification of Cassette 117
aagcgacttccaatcgctttgc

SEQ ID NO: 28 - DNA, 21 bp, Artificial Sequence
  reverse primer for amplification of Cassette 117
aaagcaaaggaaggagagaac

SEQ ID NO: 29 - DNA, 20 bp, Artificial Sequence
  forward primer for amplification of the KanMX marker Cassette
ttgcccatcgaacgtacaag

SEQ ID NO: 30 - DNA, 23 bp, Artificial Sequence
  reverse primer for amplification of the KanMX marker Cassette
tgctaagatttgtgttcgtttgg

SEQ ID NO: 31 - DNA, 20 bp, Artificial Sequence
  forward primer for amplification of the Cassette 120 and Cassette 121
cggatcgatgtacacaaccg

SEQ ID NO: 32 - DNA, 23 bp, Artificial Sequence
  reverse primer for amplification of the Cassette 120 and Cassette 121
caacaggaggcggatggatatac

SEQ ID NO: 33 - DNA, 21 bp, Artificial Sequence
  forward primer for amplification of Cassette 146, Cassette 135, Cassette 133 and Cassette 144
acgctttccggcatcttccag

SEQ ID NO: 34 - DNA, 20 bp, Artificial Sequence
  reverse primer for amplification of Cassette 146, Cassette 135, Cassette 133 and Cassette 144
gcggaatattggcggaacgg

SEQ ID NO: 35 - DNA, 22 bp, Artificial Sequence
  forward primer for amplification of Cassette 136
aacgttgtccaggtttgtatcc

SEQ ID NO: 36 - DNA, 20 bp, Artificial Sequence
  reverse primer for amplification of Cassette 136
aggtacaacaagcacgaccg

SEQ ID NO: 37 - DNA, 23 bp, Artificial Sequence
  forward primer for amplification of Cassette 137 and Cassette 139
aaataaccacaaacatccttccc

SEQ ID NO: 38 - DNA, 23 bp, Artificial Sequence
  reverse primer for amplification of Cassette 137 and Cassette 139
gtgcactacatcttgtgggtgtc

SEQ ID NO: 39 - DNA, 22 bp, Artificial Sequence
  forward primer for amplification of Cassette 140
gaaaccttcgaatccagccagc

SEQ ID NO: 40 - DNA, 24 bp, Artificial Sequence
  reverse primer for amplification of Cassette 140
acttagtatggtctgttggaaagg

SEQ ID NO: 41 - DNA, 90 bp, Artificial Sequence
  forward primer for amplification of the 3 integration flank INT1
agaaagcctgtatgcgaagccacaatcctttccaacagaccatactaagtattttattttactttttttagaatgacctgttcccgacac

SEQ ID NO: 42 - DNA, 24 bp, Artificial Sequence
  reverse primer for amplification of the 3 integration flank INT1
cacaagcttattcttccaaaaatc

SEQ ID NO: 43 - DNA, 2221 bp, Artificial Sequence
  sequence of cassette 117
aagcgacttccaatcgctttgcatatccagtaccacacccacaggcgtttgtgcaacatatatacacaattacagtaacaataacaagaggacagatactaccaaaatgtgtggggaagcgggtaagctgccacagcaattaatgcacaacatttaacctacattcttccttatcggatcctcaaaacccttaaaaacatatgcctcaccctaacatattttccaattaaccctcaatatttctctgtcacccggcctctattttccattttcttctttacccgccacgcgtttttttctttcaaatttttttcttctttcttctttttcttccacgtcctcttgcataaataaataaaccgttttgaaaccaaactcgcctctctctctcctttttgaaatatttttgggtttgtttgatcctttccttcccaatctctcttgtttaatatatattcatttatatcacgctctctttttatcttcctttttttcctctctcttgtattcttccttcccctttctactcaaaccaagaagaaaaagaaaaggtcaatctttgttaaagaataggatcttctactacatcagcttttagatttttcacgcttactgcttttttcttcccaagatcgaaaatttactgaattaacaatgggtcacggtgacactgaatctccaaacccaaccaccaccactgaaggttctggtcaaaacgaacctgaaaagaagggtcgtgacattccattatggagaaagtgtgttatcactttcgttgtttcctggatgactttggttgtcactttctcctccacctgtttgttgccagctgctccagaaattgctaacgaattcgatatgaccgtcgaaaccattaacatttccaacgctggtgttttggttgccatgggttactcttctttgatctggggtccaatgaacaaattggttggtagaagaacctcttacaacttggccatctccatgttgtgtgcctgttctgctggtactgctgctgccatcaacgaagaaatgttcattgctttccgtgtcttgtctggcttgaccggtacttctttcatggtttccggtcaaaccgtcttggctgatatctttgaaccagtttacagaggtactgctgtcggtttcttcatggctggtactctatccggtccagccattggtccatgtgtcggtggtgtcattgtcactttcacctcctggagagttatcttctggttacaattgggtatgtctggtttaggtttggttttgtctctattattcttcccaaagatcgaaggtaactctgaaaaggtttctactgctttcaagccaaccactttggtcaccatcatctccaagttctctccaaccgatgtcttgaagcaatgggtttacccaaatgtctttttggctgatttgtgttgtggtttgttggccatcactcaatactccatcttgacttctgccagagctatcttcaactccagattccatttgaccaccgctttggtttccggtttattctacttggctccaggtgctggtttcttgattggttctttggttggtggtaaattgtctgacagaaccgtcagaagatacattgtcaagagaggtttcagattacctcaagacagattgcactctggtttgatcactttgtttgctgtcttgccagctggtactttgatctacggttggactttgcaagaggacaagggtgacatggttgttccaatcattgctgctttctttgctggttggggtttgatgggttctttcaactgtttgaacacctacgttgctggtttattccacactttgatctacttgttcccattgtgtacctgtccacaataaagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacctcttgcccatcgaacgtacaagtactcctctgttctctccttcctttgcttt

SEQ ID NO: 44 - DNA, 1671 bp, Artificial Sequence
  sequence of KANMX cassette
ttgcccatcgaacgtacaagtactcctctgttctctccttcctttgctttcgtacgctgcaggtcgacgaattctaccgttcgtataatgtatgctatacgaagttatagatctgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccagtgtcgaaaacgagctcataacttcgtataatgtatgctatacgaacggtagaattcgatatcagatccactagtggcctacggatcgatgtacacaaccgactgcacccaaacgaacacaaatcttagca

SEQ ID NO: 45 - DNA, 2485 bp, Artificial Sequence
  sequence of cassette 20
cggatcgatgtacacaaccgactgcacccaaacgaacacaaatcttagcagtgcttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgaccaagcaatctgctgactccaatgccaagtctggtgttacttctgaaatctgtcactgggcttctaacttggctaccgatgacatcccatctgatgtcttggaaagagctaagtacttgatcttggacggtattgcttgtgcttgggttggtgccagagttccatggtctgaaaagtacgttcaagctaccatgtccttcgaacctccaggtgcttgtcgtgtcattggttacggtcaaaaattgggtcctgttgctgctgccatgaccaactctgcctttattcaagctactgaattggacgactaccactctgaagctccattacattccgcttccattgtcttaccagctgtctttgctgcttctgaagttttggctgaacaaggtaagactatctctggtatcgatgtcatcttggctgccattgtcggtttcgaatccggtccaagaatcggtaaggccatctacggttccgatttgttgaacaacggttggcattgtggtgccgtttacggtgccccagctggtgctttggctaccggtaagctattaggtttgactccagactccatggaagatgctttgggtattgcctgtacccaagcttgtggtttgatgtccgctcaatacggtggtatggtcaagagagtccaacacggtttcgctgccagaaacggtttgttgggtggtttgttggctcacggtggttacgaagctatgaagggtgttttggaaagatcttacggtggtttcttgaagatgttcaccaagggtaacggtagagaaccaccatacaaggaagaagaagttgttgctggtttaggttctttctggcacactttcaccatcagaatcaaattgtacgcttgttgtggtttagtccacggtccagttgaagccatcgaaaacttgcaaggtagatacccagaattattgaacagagctaacttgtccaacatcagacacgttcacgttcaattgtccactgcttctaactctcactgtggttggatcccagaagaaagaccaatttcttccattgctggtcaaatgtccgttgcttacattttggctgttcaattggttgaccaacaatgtttgttgtctcaattctctgaattcgatgacaacttggaaagaccagaagtctgggacttggccagaaaggttacctcttctcaatctgaagaattcgaccaagatggtaactgtctatccgctggtcgtgtcagaatcgaattcaacgacggttcttccatcactgaatctgttgaaaagccattgggtgtcaaggaaccaatgccaaacgaaagaattttgcacaaatacagaactttggctggttccgtcactgacgaatccagagtcaaggaaattgaagatttggttttgggtttagatcgtttgactgacatctctccattattggaattgttgaactgtccagtcaaatctccattcgggatctaaaataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgccttcctcacgctttccggcatcttccagaccacagtatatccatccgcctcctgttg

SEQ ID NO: 46 - DNA, 2488 bp, Artificial Sequence
  sequence of cassette 121
cggatcgatgtacacaaccgactgcacccaaacgaacacaaatcttagcagtgcttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgaccaagcaatctgctgactccaatgccaagtctggtgtcacttccgaaatctgtcactgggcttccaacttggctactgacgacattccatctgatgtcttggaaagagccaagtacttgattttggacggtattgcctgtgcttgggttggtgctcgtgttccatggtctgaaaagtacgttcaagctaccatgtccttcgaacctccaggtgcttgtcgtgtcatcggttacggtcaaaaattgggtccagttgctgctgccatgaccaactctgcctttattcaagccactgaattggatgactaccactctgaagctccattgcactctgcttccattgttctaccagctgttttcgctgcttctgaagtcttggctgaacaaggtaagaccatctctggtatcgatgttatcttagctgccattgtcggtttcgaatctggtccaagaatcggtaaggccatctacggttctgacttgttgaacaacggttggcattgtggtgccgtttacggtgctccagctggtgctttggctaccggtaagttgttgggtttgactccagactccatggaagatgctttgggtatcgcttgtacccaagcttgtggtttgatgtctgctcaatacggtggtatggttaagagagttcaacatggtttcgctgccagaaacggtctattaggtggtttgttggctcacggtggttacgaagctatgaagggtgtcttggaaagatcttacggtggtttcttgaagatgttcaccaagggtaacggtagagaacctccatacaaggaagaagaagttgttgccggtttaggttctttctggcacactttcaccatcagaatcaaattgtacgcttgttgtggtttagtccacggtccagttgaagccattgaaaacttacaaggtcgttacccagaattgttgaacagagctaacttgtccaacatcagacacgttcacgttcaattatccactgcttccaactctcactgtggttggattccagaagaaagaccaatctcctccattgctggtcaaatgtctgttgcttacattttggctgtccaattggttgaccaacaatgtttgttgtctcaattctccgaattcgatgacaacttggaaagaccagaagtctgggatttggctagaaaggtcacctcttctcaatctgaagaatttgaccaagatggtaactgtttgtctgctggtagagtcagaattgaattcaacgacggttcttccatcactgaatccgttgaaaagccattaggtgtcaaggaaccaatgccaaacgaaagaatcttgcacaaatacagaactttggctggttccgtcactgacgaatccagagtcaaggaaatcgaagatttggttttgggtttggacagattgaccgatatctctccattattggaattgttgaactgtccagtcaaatctccattgggtatcaagtaaaataaagcaatcttgatgaggataatgatttttttttgaatatacataaatactaccgtttttctgctagattttgtgaagacgtaaataagtacatattactttttaagccaagacaagattaagcattaactttacccttttctcttctaagtttcaatactagttatcactgtttaaaagttatggcgagaacgtcggcggttaaaatatattaccctgaacgtggtgaattgaagttctaggatggtttaaagatttttcctttttgggaaataagtaaacaatatattgctgccttcctcacgctttccggcatcttccagaccacagtatatccatccgcctcctgttg

SEQ ID NO: 47 - DNA, 3298 bp, Artificial Sequence
  sequence of cassette 133
acgctttccggcatcttccagaccacagtatatccatccgcctcctgttggtgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgactgtttccaacttgaccagagactccaaggttaaccaaaacttgttggaagatcattctttcatcaactacaagcaaaatgtcgaaactttggatatcgtcagaaagagattgaacagaccattcacctacgctgaaaagattttgtacggtcacttggatgacccacacggtcaagatatccaaagaggtgtctcctacttgaaactaagaccagatcgtgttgcttgtcaagatgctactgctcaaatggctatcttacaattcatgtccgctggtttgcctcaagttgccaagccagtcaccgtccactgtgaccatttgatccaagctcaagtcggtggtgaaaaggacttgaagagagccattgacttgaacaaggaagtctacgacttcttggcttctgccactgctaaatacaacatgggtttctggaagccaggttccggtatcatccaccaaatcgttttggaaaactatgccttcccaggtgctttgatcatcggtactgactcccacactccaaatgccggtggtctaggtcaattggccatcggtgttggtggtgctgatgctgttgacgtcatggctggtagaccatgggaattgaaggctccaaagattttgggtgttaagttgaccggtaagatgaacggttggacttctccaaaggacatcatcttgaaattggctggtatcactactgttaagggtggtactggtaagattgtcgaatactttggtgacggtgtcgacactttctctgctaccggtatgggtaccatctgtaacatgggtgctgaaattggtgccaccacttctgttttcccattcaacaaatccatgattgaatacttggaagctaccggtagaggtaagattgctgatttcgctaagttataccacaaggacttgttgtctgccgacaaggacgctgaatacgatgaagttgtcgaaattgacttgaacactttggaaccatacatcaacggtccattcaccccagatttggctaccccagtttctaagatgaaggaagttgccgttgctaacaactggccattagatgttagagttggtttgattggttcttgtaccaactcctcttacgaagatatgtccagatctgcttccattgtcaaggatgctgctgctcacggtttgaaatctaagaccatcttcactgttaccccaggttctgaacaaatcagagccaccatcgaacgtgacggtcaattggaaactttcaaggaatttggtggtattgtcttggctaacgcttgtggtccatgtattggtcaatgggacagaagagatatcaagaagggtgacaagaacaccatcgtttcctcttacaacagaaacttcacttctagaaacgatggtaacccacaaacccacgcctttgttgcttctccagaattagtcactgctttcgctattgctggtgacttgagattcaacccattaaccgacaaattgaaggacaaggacggtaacgaatttatgttgaagcctcctcatggtgatggtttaccacaaagaggttacgatgctggtgaaaacacctaccaagctccaccagccgacagatccaccgtcgaagtcaaggtttctccaacttctgacagattacaattgttgaaacctttcaagccatgggatggtaaggacgctaaggacatgccaatcttaatcaaggctgttggtaagactaccaccgaccacatttccatggctggtccatggttgaaatacagaggtcacttggaaaacatctccaacaactacatgattggtgccattaatgccgaaaacaagaaggctaactgtgtcaagaacgtttacactggtgaatacaagggtgttccagacactgccagagactacagagatcaaggtatcaaatgggttgtcatcggtgacgaaaacttcggtgaaggttcttctcgtgaacacgctgctttggaaccaagattcttgggtggtttcgctattattaccaaatctttcgctcgtattcacgaaaccaacttgaagaagcaaggtctattgccattgaacttcaagaacccagccgactacgacaagatcaacccagatgacagaattgacatcttaggtttggctgaattggctccaggtaagccagtcaccatgagagttcacccaaagaacggtaagccatgggatgctgtcttgactcacactttcaacgatgaacaaatcgaatggttcaaatacggttctgctttgaacaagatcaaggctgatgaaaagaagtaaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatacctcaacgttgtccaggtttgtatccacgtgtgtccgttccgccaatattccgc

SEQ ID NO: 48 - DNA, 3607 bp, Artificial Sequence
  sequence of cassette 135
acgctttccggcatcttccagaccacagtatatccatccgcctcctgttggtgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaagacaaagacataatacttctctatcaatttcagttattgttcttccttgcgttattcttctgttcttctttttcttttgtcatatataaccataaccaagtaatacatattcaaaatgttggaagaatacagaaagcatgttgctgaaagagctgctgaaggtattgctccaaagccattggacgctaaccaaatggccgctttggttgaattgttgaagaacccaccagccggtgaagaagaattcttgttggatttgttgaccaacagagttcctcctggtgttgacgaagccgcttacgtcaaggctggtttcttggctgccattgccaagggtgaagctaagtctcctttgttgaccccagaaaaggccatcgaattattgggtaccatgcaaggtggttacaacattcacccattgattgacgctctagacgatgctaagttggctccaattgctgccaaggctctatcccacactttgttgatgttcgacaacttctacgatgtcgaagaaaaggccaaggccggtaacgaatacgctaagcaagttatgcaatcctgggctgatgctgaatggttcttgaacagaccagctttggctgaaaaattgactgtcaccgttttcaaggtcactggtgaaaccaacaccgatgacttgtctccagctccagatgcttggtccagaccagatatcccattgcacgctttggccatgttgaaaaatgctcgtgaaggtattgaaccagaccaaccaggtgttgtcggtccaatcaagcaaatcgaagctttgcaacaaaaaggtttcccattggcttacgtcggtgatgttgtcggtaccggttcttccagaaagtctgctaccaactctgttttatggttcatgggtgatgatatcccacacgttccaaacaagagaggtggtggtttgtgtttgggtggtaagatcgccccaattttcttcaacaccatggaagatgccggtgctttgccaattgaagtcgatgtctccaacttgaacatgggtgacgtcattgatgtttacccatacaagggtgaagtcagaaaccacgaaactggtgaattgttggctacctttgaattaaagactgacgtcttgattgacgaagtcagagctggtggtagaatcccattgatcatcggtagaggtttgactaccaaggccagagaagctttaggtttgcctcactccgatgttttcagacaagctaaggatgtcgctgaatctgacagaggtttctccttggcccaaaagatggttggtagagcttgtggtgtcaagggtatcagaccaggtgcttactgtgaaccaaagatgacttccgttggttctcaagacaccactggtccaatgaccagagatgaattgaaggacttggcttgtttgggtttctccgctgacttggttatgcaatctttctgtcacactgctgcttacccaaagccagttgacgtcaacacccatcacactctaccagacttcatcatgaaccgtggtggtgtttctttgcgtccaggtgacggtgtcattcactcctggttaaacagaatgttgttgccagacaccgttggtaccggtggtgactctcacacccgtttcccaatcggtatttctttcccagccggttccggtttggttgcctttgctgccgctactggtgtcatgccattagacatgccagaatctgttttggtcagattcaagggtaagatgcaaccaggtatcactttgagagacttagtccacgctatcccattatacgccatcaagcaaggtttgttgactgtcgaaaagaagggtaagaaaaatattttctctggtcgtattttggaaatcgaaggtttgccagatttgaaggtcgaacaagcctttgaattgactgatgcttctgctgaaagatctgccgctggttgtaccatcaaattgaacaaggaacctatcatcgaatacttgaactccaacattgtcttattgaaatggatgattgctgaaggttacggtgacagaagaactttggaaagaagaatccaaggtatggaaaaatggttagctaacccagaattgttggaagctgacgctgatgctgaatacgctgctgttatcgatatcgatttggctgacatcaaggaaccaatcctatgtgccccaaatgacccagatgacgctagaccattatctgctgtccaaggtgaaaagattgacgaagtctttatcggttcttgtatgaccaacatcggtcatttcagagctgctggtaagttgttggacgctcacaagggtcaattgccaaccagattatgggttgccccaccaactagaatggacgctgctcaattgaccgaagaaggttactactctgttttcggtaaatctggtgcccgtattgaaattccaggttgttccttgtgtatgggtaaccaagctagagttgctgacggtgctaccgttgtttccacttctaccagaaacttcccaaacagattaggtactggtgccaacgttttcttggcttctgctgaattggctgctgttgctgctttgatcggtaaattgccaactccagaagaataccaaacttacgttgctcaagtcgacaagactgctgttgacacctacagatacttgaacttcaaccaattgtctcaatacactgaaaaggctgacggtgttatcttccaaactgcggtttaaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatacctcaacgttgtccaggtttgtatccacgtgtgtccgttccgccaatattccgc

SEQ ID NO: 49 - DNA, 3298 bp, Artificial Sequence
  sequence of cassette 144
acgctttccggcatcttccagaccacagtatatccatccgcctcctgttggtgccaaacattaatttgttctgcatactttgaacctttcagaaaataaaaaacattacgcgcatacttaccctgctcgcgaagaagagtaacactaacgcattctatgggcaattgaagacagtattcagtacaagacatagtccgtttccttgagtcaattcctatagcattatgaactagccgcctttaagagtgccaagctgttcaacaccgatcatttttgatgatttggcgtttttgttatattgatagatttcttttgaattttgtcattttcacttttccactcgcaacggaatccggtggcaaaaaagggaaaagcattgaaatgcaatctttaacagtattttaaacaagttgcgacacggtgtacaattacgataagaattgctacttcaaagtacacacagaaagttaacatgaatggaattcaagtggacatcaatcgtttgaaaaagggcgaagtcagtttaggtacctcaatgtatgtatataagaatttttcctcccactttattgtttctaaaagttcaatgaagtaaagtctcaattggccttattactaactaataggtatcttataatcacctaataaaatagaatgactgtttccaacttgaccagagactccaaggttaaccaaaacttgttggaagatcattctttcatcaactacaagcaaaatgtcgaaactttggatatcgtcagaaagagattgaacagaccattcacctacgctgaaaagattttgtacggtcacttggatgacccacacggtcaagatatccaaagaggtgtctcctacttgaaactaagaccagatcgtgttgcttgtcaagatgctactgctcaaatggctatcttacaattcatgtccgctggtttgcctcaagttgccaagccagtcaccgtccactgtgaccatttgatccaagctcaagtcggtggtgaaaaggacttgaagagagccattgacttgaacaaggaagtctacgacttcttggcttctgccactgctaaatacaacatgggtttctggaagccaggttccggtatcatccaccaaatcgttttggaaaactatgccttcccaggtgctttgatcatcggtactgactcccacactccaaatgccggtggtctaggtcaattggccatcggtgttggtggtgctgatgctgttgacgtcatggctggtagaccatgggaattgaaggctccaaagattttgggtgttaagttgaccggtaagatgaacggttggacttctccaaaggacatcatcttgaaattggctggtatcactactgttaagggtggtactggtaagattgtcgaatactttggtgacggtgtcgacactttctctgctaccggtatgggtaccatctgtaacatgggtgctgaaattggtgccaccacttctgttttcccattcaacaaatccatgattgaatacttggaagctaccggtagaggtaagattgctgatttcgctaagttataccacaaggacttgttgtctgccgacaaggacgctgaatacgatgaagttgtcgaaattgacttgaacactttggaaccatacatcaacggtccattcaccccagatttggctaccccagtttctaagatgaaggaagttgccgttgctaacaactggccattagatgttagagttggtttgattggttcttgtaccaactcctcttacgaagatatgtccagatctgcttccattgtcaaggatgctgctgctcacggtttgaaatctaagaccatcttcactgttaccccaggttctgaacaaatcagagccaccatcgaacgtgacggtcaattggaaactttcaaggaatttggtggtattgtcttggctaacgcttgtggtccatgtattggtcaatgggacagaagagatatcaagaagggtgacaagaacaccatcgtttcctcttacaacagaaacttcacttctagaaacgatggtaacccacaaacccacgcctttgttgcttctccagaattagtcactgctttcgctattgctggtgacttgagattcaacccattaaccgacaaattgaaggacaaggacggtaacgaatttatgttgaagcctcctcatggtgatggtttaccacaaagaggttacgatgctggtgaaaacacctaccaagctccaccagccgacagatccaccgtcgaagtcaaggtttctccaacttctgacagattacaattgttgaaacctttcaagccatgggatggtaaggacgctaaggacatgccaatcttaatcaaggctgttggtaagactaccaccgaccacatttccatggctggtccatggttgaaatacagaggtcacttggaaaacatctccaacaactacatgattggtgccattaatgccgaaaacaagaaggctaactgtgtcaagaacgtttacactggtgaatacaagggtgttccagacactgccagagactacagagatcaaggtatcaaatgggttgtcatcggtgacgaaaacttcggtgaaggttcttctcgtgaacacgctgctttggaaccaagattcttgggtggtttcgctattattaccaaatctttcgctcgtattcacgaaaccaacttgaagaagcaaggtctattgccattgaacttcaagaacccagccgactacgacaagatcaacccagatgacagaattgacatcttaggtttggctgaattggctccaggtaagccagtcaccatgagagttcacccaaagaacggtaagccatgggatgctgtcttgactcacactttcaacgatgaacaaatcgaatggttcaaatacggttctgctttgaacaagatcaaggctgatgaaaagaagtaaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatacctcaacgttgtccaggtttgtatccacgtgtgtccgttccgccaatattccgc

SEQ ID NO: 50 - DNA, 3607 bp, Artificial Sequence
  sequence of cassette 146
acgctttccggcatcttccagaccacagtatatccatccgcctcctgttggtgccaaacattaatttgttctgcatactttgaacctttcagaaaataaaaaacattacgcgcatacttaccctgctcgcgaagaagagtaacactaacgcattctatgggcaattgaagacagtattcagtacaagacatagtccgtttccttgagtcaattcctatagcattatgaactagccgcctttaagagtgccaagctgttcaacaccgatcatttttgatgatttggcgtttttgttatattgatagatttcttttgaattttgtcattttcacttttccactcgcaacggaatccggtggcaaaaaagggaaaagcattgaaatgcaatctttaacagtattttaaacaagttgcgacacggtgtacaattacgataagaattgctacttcaaagtacacacagaaagttaacatgaatggaattcaagtggacatcaatcgtttgaaaaagggcgaagtcagtttaggtacctcaatgtatgtatataagaatttttcctcccactttattgtttctaaaagttcaatgaagtaaagtctcaattggccttattactaactaataggtatcttataatcacctaataaaatagaatgttggaagaatacagaaagcatgttgctgaaagagctgctgaaggtattgctccaaagccattggacgctaaccaaatggccgctttggttgaattgttgaagaacccaccagccggtgaagaagaattcttgttggatttgttgaccaacagagttcctcctggtgttgacgaagccgcttacgtcaaggctggtttcttggctgccattgccaagggtgaagctaagtctcctttgttgaccccagaaaaggccatcgaattattgggtaccatgcaaggtggttacaacattcacccattgattgacgctctagacgatgctaagttggctccaattgctgccaaggctctatcccacactttgttgatgttcgacaacttctacgatgtcgaagaaaaggccaaggccggtaacgaatacgctaagcaagttatgcaatcctgggctgatgctgaatggttcttgaacagaccagctttggctgaaaaattgactgtcaccgttttcaaggtcactggtgaaaccaacaccgatgacttgtctccagctccagatgcttggtccagaccagatatcccattgcacgctttggccatgttgaaaaatgctcgtgaaggtattgaaccagaccaaccaggtgttgtcggtccaatcaagcaaatcgaagctttgcaacaaaaaggtttcccattggcttacgtcggtgatgttgtcggtaccggttcttccagaaagtctgctaccaactctgttttatggttcatgggtgatgatatcccacacgttccaaacaagagaggtggtggtttgtgtttgggtggtaagatcgccccaattttcttcaacaccatggaagatgccggtgctttgccaattgaagtcgatgtctccaacttgaacatgggtgacgtcattgatgtttacccatacaagggtgaagtcagaaaccacgaaactggtgaattgttggctacctttgaattaaagactgacgtcttgattgacgaagtcagagctggtggtagaatcccattgatcatcggtagaggtttgactaccaaggccagagaagctttaggtttgcctcactccgatgttttcagacaagctaaggatgtcgctgaatctgacagaggtttctccttggcccaaaagatggttggtagagcttgtggtgtcaagggtatcagaccaggtgcttactgtgaaccaaagatgacttccgttggttctcaagacaccactggtccaatgaccagagatgaattgaaggacttggcttgtttgggtttctccgctgacttggttatgcaatctttctgtcacactgctgcttacccaaagccagttgacgtcaacacccatcacactctaccagacttcatcatgaaccgtggtggtgtttctttgcgtccaggtgacggtgtcattcactcctggttaaacagaatgttgttgccagacaccgttggtaccggtggtgactctcacacccgtttcccaatcggtatttctttcccagccggttccggtttggttgcctttgctgccgctactggtgtcatgccattagacatgccagaatctgttttggtcagattcaagggtaagatgcaaccaggtatcactttgagagacttagtccacgctatcccattatacgccatcaagcaaggtttgttgactgtcgaaaagaagggtaagaaaaatattttctctggtcgtattttggaaatcgaaggtttgccagatttgaaggtcgaacaagcctttgaattgactgatgcttctgctgaaagatctgccgctggttgtaccatcaaattgaacaaggaacctatcatcgaatacttgaactccaacattgtcttattgaaatggatgattgctgaaggttacggtgacagaagaactttggaaagaagaatccaaggtatggaaaaatggttagctaacccagaattgttggaagctgacgctgatgctgaatacgctgctgttatcgatatcgatttggctgacatcaaggaaccaatcctatgtgccccaaatgacccagatgacgctagaccattatctgctgtccaaggtgaaaagattgacgaagtctttatcggttcttgtatgaccaacatcggtcatttcagagctgctggtaagttgttggacgctcacaagggtcaattgccaaccagattatgggttgccccaccaactagaatggacgctgctcaattgaccgaagaaggttactactctgttttcggtaaatctggtgcccgtattgaaattccaggttgttccttgtgtatgggtaaccaagctagagttgctgacggtgctaccgttgtttccacttctaccagaaacttcccaaacagattaggtactggtgccaacgttttcttggcttctgctgaattggctgctgttgctgctttgatcggtaaattgccaactccagaagaataccaaacttacgttgctcaagtcgacaagactgctgttgacacctacagatacttgaacttcaaccaattgtctcaatacactgaaaaggctgacggtgttatcttccaaactgcggtttaaagtctgaagaatgaatgatttgatgatttctttttccctccatttttcttactgaatatatcaatgatatagacttgtatagtttattatttcaaattaagtagctatatatagtcaagataacgtttgtttgacacgattacattattcgtcgacatcttttttcagcctgtcgtggtagcaatttgaggagtattattaattgaataggttcattttgcgctcgcataaacagttttcgtcagggacagtatgttggaatgagtggtaattaatggtgacatgacatgttatagcaatacctcaacgttgtccaggtttgtatccacgtgtgtccgttccgccaatattccgc

SEQ ID NO: 51 - DNA, 4552 bp, Artificial Sequence
  sequence of cassette 136
aacgttgtccaggtttgtatccacgtgtgtccgttccgccaatattccgcgtgcgggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgtcctcttccaagatcttggctggtttgagagacaacttttctttgttgggtgaaaagaacaagattttggtcgccaacagaggtgaaatcccaatcagaattttcagatctgctcacgaattgtctatgagaactatcgccatctactctcacgaagatagattatccatgcacagattgaaggctgatgaagcctacgttatcggtgaagaaggtcaatacaccccagtcggtgcttacttggccatggacgaaatcatcgaaattgccaagaagcacaaggtcgatttcatccacccaggttacggtttcttgtctgaaaactctgaatttgctgacaaggttgttaaggctggtattacctggattggtccaccagctgaagtcattgaatctgttggtgacaaggtttctgccagacatttggctgctcgtgccaacgttccaactgtcccaggtactccaggtcctatcgaaaccgttcaagaagctctagatttcgtcaatgaatacggttacccagttatcatcaaggctgctttcggtggtggtggtcgtggtatgagagttgtcagagaaggtgacgatgtcgctgatgctttccaaagagccacttctgaagctagaactgctttcggtaacggtacttgtttcgtcgaaagattcttggacaagccaaagcacattgaagttcaattattagctgacaaccacggtaacgttgtccacttgttcgaaagagactgttccgtccaaagacgtcaccaaaaggttgtcgaagttgctccagctaagactttaccaagagaagttagagatgctatcttgaccgatgccgttaagttggctaaggtttgtggttacagaaacgctggtactgctgaattcttggttgacaaccaaaacagacattacttcattgaaatcaacccaagaattcaagtcgaacacaccatcactgaagaaatcactggtattgacattgtctccgctcaaatccaaatcgccgctggtgctactttgactcaattaggtctattacaagacaaaatcaccaccagaggtttctctatccaatgtcgtatcaccactgaagatccatccaagaacttccaaccagacactggtcgtttggaagtctacagatccgctggtggtaacggtgtcagattggacggtggtaacgcctacgctggtgctaccatctctccacactacgactccatgttggttaagtgttcctgttctggttctacctacgaaattgtcagaagaaagatgatcagagctttgattgaattcagaatcagaggtgtcaagaccaacatcccattcttgttgactttgttgaccaacccagttttcattgaaggtacctactggaccactttcatcgatgacactccacaattgttccaaatggtttcctctcaaaacagagctcaaaaattgttgcactacttggctgacttggccgtcaacggttcctctatcaagggtcaaatcggtttaccaaagttgaagtccaacccttccgttccacatttgcacgatgctcaaggtaatgtcatcaacgttaccaaatctgccccaccatccggttggagacaagtcttgttggaaaagggtccatccgaatttgccaagcaagtcagacaattcaacggtactttgttgatggacaccacctggagagatgctcaccaatctttgctagctaccagagtcagaactcacgatttggccaccattgctccaaccactgctcacgctttggctggtgcctttgctttggaatgttggggtggtgctactttcgatgtcgccatgagattcttgcatgaggacccatgggaaagattgagaaaattgagatctttggtcccaaacattccattccaaatgttgttgagaggtgctaacggtgttgcttactcctctttgccagacaacgccattgaccatttcgttaagcaagccaaggacaatggtgttgacattttcagagtctttgacgctttgaacgacttggaacaattgaaggttggtgttaatgctgtcaagaaggctggtggtgttgtcgaagctaccgtttgttactctggtgacatgttgcaaccaggtaagaaatacaacttggactactacttagaagttgtcgaaaagatcgttcaaatgggtactcacatcttgggtatcaaggacatggctggtaccatgaagccagctgctgccaaattgttgattggttctttacgtaccagatacccagacttgccaatccacgttcactctcatgactccgctggtactgctgttgcttccatgactgcttgtgctttggccggtgctgatgttgttgacgttgccattaactccatgtccggtttgacctctcaaccatctattaacgctttgttggcctccttggaaggtaacattgacactggtatcaacgtcgaacacgttagagaattggacgcttactgggctgaaatgagattattatactcttgtttcgaagctgacttgaagggtccagaccctgaagtttaccaacacgaaattccaggtggtcaattgaccaacttgttgttccaagctcaacaattaggtctaggtgaacaatgggctgaaaccaagagagcttacagagaagctaactacttgttgggtgacattgttaaggtcaccccaacttctaaggtcgttggtgatttggctcaattcatggtttctaacaaattgacttctgatgacatcagaagattagctaactctttggacttcccagactccgttatggacttcttcgaaggtttgatcggtcaaccatacggtggtttcccagaaccattgagatccgatgttttgagaaacaagcgtcgtaaattgacttgtagaccaggtttagaattggaaccattcgatttggaaaagatcagagaagatttgcaaaacagattcggtgatatcgatgaatgtgatgttgcctcctacaacatgtatcctcgtgtctacgaagatttccaaaagattagagaaacttacggtgacttgtctgtcttaccaaccaagaacttcttggctccagctgaaccagacgaagaaatcgaagtcaccattgaacaaggtaagactttgattatcaaattacaagctgttggtgatttgaacaagaaaaccggtcaaagagaagtctacttcgaattgaacggtgaattgagaaagatcagagttgctgacaaatctcaaaacattcaatctgttgccaagccaaaggctgatgtccacgacacccaccaaatcggtgctccaatggctggtgtcatcattgaagtcaaggttcacaagggttctttggtcaagaagggtgaatctatcgccgttttgtctgctatgaagatggaaatggttgtttcctctccagctgatggtcaagtcaaagatgtctttatccgtgacggtgaatccgtcgatgcttctgacttgttggttgttttggaagaagaaactctaccaccttctcaaaagaaataaagattaatataattatataaaaatattatcttcttttctttatatctagtgttatgtaaaataaattgatgactacggaaagcttttttatattgtttctttttcattctgagccacttaaatttcgtgaatgttcttgtaagggacggtagatttacaagtgatacaacaaaaagcaaggcgctttttctaataaaaagaagaaaagcatttaacaattgaacacctctatatcaacgaagaatattactttgtctctaaatccttgtaaaatgtgtacgatctctatatgggttactcacctcaaataaccacaaacatccttcccatatgctcggtcgtgcttgttgtacct

SEQ ID NO: 52 - DNA, 2341 bp, Artificial Sequence
  sequence of cassette 137
aaataaccacaaacatccttcccatatgctcggtcgtgcttgttgtacctgtgcttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatgtcctctgcttctgaacaaactttgaaggaaagatttgctgaaatcattccagctaaggctgaagaaatcaagaaattcaagaaggaacacggtaagactgttatcggtgaagtcttgttggaacaagcttacggtggtatgagaggtatcaagggtttagtctgggaaggttctgttttggacccagaagaaggtatcagattccgtggtagaaccattccagaaatccaaagagaattgccaaaggctgaaggttccactgaaccattaccagaagctttgttctggttattgttgaccggtgaaattccaaccgatgctcaagtcaaggctttgtctgctgatttggctgcccgttctgaaatcccagaacacgttatccaattgttggactctctaccaaaggacttgcacccaatggctcaattctccattgctgttaccgccttggaatctgaatccaagttcgctaaggcctacgctcaaggtgtttccaagaaggaatactggtcctacaccttcgaagattctttggatttgttgggtaaattgcctgtcattgcttccaagatctacagaaacgttttcaaggacggtaagatcacttctactgacccaaacgctgactacggtaagaacttggctcaattgttgggttacgaaaacaaagatttcatcgatttgatgagattatacttgaccattcactctgaccacgaaggtggtaatgtctctgctcacactacccacttggtcggttctgctttgtcctctccatacttgtctttggctgccggtttgaacggtttggctggtcctttgcacggtagagctaaccaagaagtcttggaatggttattcaaattgagagaagaagtcaagggtgactactccaaggaaaccattgaaaaatacttatgggacactttgaacgccggtcgtgttgttccaggttacggtcatgccgttttgagaaagaccgatccaagatacactgcccaaagagaatttgctttgaagcatttcccagactacgaattattcaaattggtttccaccatctacgaagttgctccaggtgtcttgaccaagcacggtaagaccaagaacccatggccaaacgttgactctcactctggtgttttgctacaatactacggtttgactgaagcttctttctacactgtcttattcggtgttgccagagccattggtgtcttgccacaattgatcattgacagagctgttggtgctccaattgaaagaccaaagtctttctccactgaaaaatacaaggaattggtcaagaagatcgaatccaagaactaaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacacctcgaaaccttcgaatccagccagcatgtcgacacccacaagatgtagtgcac

SEQ ID NO: 53 - DNA, 2293 bp, Artificial Sequence
  sequence of cassette 139
aaataaccacaaacatccttcccatatgctcggtcgtgcttgttgtacctgtgcttggctgataatagcgtataaacaatgcatactttgtacgttcaaaatacaatgcagtagatatatttatgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagacaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaatggctgacaccaaggccaagttgaccttgaacggtgacactgctgtcgaattggatgttttgaaaggtactttgggtcaagatgtcattgatatcagaactttgggttccaagggtgttttcaccttcgacccaggtttcacctctactgcttcttgtgaatccaagatcactttcatcgatggtgacgaaggtatcctattacacagaggtttcccaattgaccaattagctactgactccaactacttggaagtttgttacatcttgttgaatggtgaaaagccaactcaagaacaatacgacgaatttaaaaccaccgttaccagacacaccatgattcacgaacaaatcaccagattattccacgctttccgtcgtgactcccacccaatggctgtcatgtgtggtatcactggtgctttggctgctttctaccatgactctttggatgtcaacaacccaagacacagagaaattgccgctttcttgttgttgtccaagatgccaaccatggctgctatgtgttacaagtactccatcggtcaacctttcgtttacccaagaaacgatttgtcttacgccggtaacttcttgaacatgatgttctccactccatgtgaaccttacgaagttaacccaattttggaaagagccatggacagaatcttgatcttgcacgctgaccatgaacaaaacgcttctacttctactgttagaactgccggttcttctggtgctaacccatttgcttgtatcgctgctggtattgcttctttatggggtccagctcatggtggtgccaacgaagctgctttgaagatgttggaagaaatttcttctgtcaagcacattccagaatttgtcagaagagctaaggacaagaacgactctttcagattgatgggtttcggtcaccgtgtctacaagaactacgacccaagagctaccgtcatgagagaaacctgtcacgaagttttgaaggaattgggtaccaaggatgacttgttggaagttgccatggaattggaaaacattgctttgaacgacccatacttcatcgaaaagaaattgtacccaaacgtcgatttctactccggtatcatcttaaaggctatgggtattccatcttccatgttcaccgttatctttgctatggccagaactgttggttggatcgctcactggtccgaaatgcactctgatggtatgaagattgccagaccaagacaattatacactggttacgaaaagagagatttcaaatctgatatcaagagataaagcgatttaatctctaattattagttaaagttttataagcatttttatgtaacgaaaaataaattggttcatattattactgcactgtcacttaccatggaaagaccagacaagaagttgccgacagtctgttgaattggcctggttaggcttaagtctgggtccgcttctttacaaatttggagaatttctcttaaacgatatgtatattcttttcgttggaaaagatgtcttccaaaaaaaaaaccgatgaattagtggaaccaaggaaaaaaaaagaggtatccttgattaaggaacacctcgaaaccttcgaatccagccagcatgtcgacacccacaagatgtagtgcac

SEQ ID NO: 54 - DNA, 2419 bp, Artificial Sequence
  sequence of cassette 140
gaaaccttcgaatccagccagcatgtcgacacccacaagatgtagtgcacgtgcgtgtcgacgctgcgggtatagaaagggttctttactctatagtacctcctcgctcagcatctgcttcttcccaaagatgaacgcggcgttatgtcactaacgacgtgcaccaacttgcggaaagtggaatcccgttccaaaactggcatccactaattgatacatctacacaccgcacgccttttttctgaagcccactttcgtggactttgccatatgcaaaattcatgaagtgtgataccaagtcagcatacacctcactagggtagtttctttggttgtattgatcatttggttcatcgtggttcattaattttttttctccattgctttctggctttgatcttactatcatttggatttttgtcgaaggttgtagaattgtatgtgacaagtggcaccaagcatatataaaaaaaaaaagcattatcttcctaccagagttgattgttaaaaacgtatttatagcaaacgcaattgtaattaattcttattttgtatcttttcttcccttgtctcaatcttttatttttattttatttttcttttcttagtttctttcataacaccaagcaactaatactataacatacaataataatggaatctttggaattggaacaattagtcaagaaggttttgttggaaaaattggctgaacaaaaggaagttccaaccaagaccaccacccaaggtgccaagtccggtgttttcgacaccgtcgatgaagctgtccaagctgctgtcattgctcaaaactgttacaaggaaaaatctttggaagaaagaagaaacgttgtcaaggccatcagagaagctttgtacccagaaatcgaaaccattgccaccagagctgttgctgaaaccggtatgggtaatgtcactgacaagatcttgaagaacactttggccatcgaaaagaccccaggtgttgaagatttgtacactgaagttgccactggtgacaacggtatgactttgtacgaattgtctccatacggtgtcatcggtgctgttgccccatctaccaacccaactgaaactttgatctgtaactccattggtatgttggctgctggtaatgctgttttctactctcctcacccaggtgccaagaacatctctttatggttaatcgaaaaattgaacaccattgtccgtgactcttgtggtatcgacaacttgattgtcactgttgccaagccttccatccaagctgctcaagaaatgatgaaccatccaaaggtcccattgttggttatcactggtggtccaggtgttgtcttgcaagctatgcaatctggtaagaaggtcattggtgctggtgctggtaacccaccatctatcgtcgatgaaactgctaacattgaaaaggctgccgctgatatcgttgacggtgcttctttcgaccacaacatcctatgtattgctgaaaaatccgttgttgccgttgactccattgctgatttcttattattccaaatggaaaagaacggtgctttgcacgttaccaacccatctgatatccaaaaattggaaaaggttgctgtcactgacaagggtgtcaccaacaagaaattggttggtaagtctgctactgaaatcttgaaggaagctggtattgcttgtgacttcactccaagattaatcattgtcgaaactgaaaagtcccacccatttgccaccgttgaattgttgatgccaattgtcccagttgtcagagttccagacttcgatgaagctttggaagttgccatcgaattggaacaaggtttgcaccacactgctaccatgcactctcaaaacatctccagattgaacaaggctgctagagacatgcaaacttccatctttgtcaagaacggtccatctttcgctggtttaggtttcagaggtgaaggttccactactttcaccattgctaccccaactggtgaaggtaccaccaccgctagacatttcgctagaagaagaagatgtgttttgactgatggtttctccatacgttaaaggaagtatctcggaaatattaatttaggccatgtccttatgcacgtttcttttgatacttacgggtacatgtacacaagtatatctatatatataaattaatgaaaatcccctatttatatatatgactttaacgagacagaacagttttttattttttatcctatttgatgaatgatacagtttcttattcacgtgttatacccacaccaaatccaatagcaataccggccatcacaatcactgtttcggcagcccctaagatcagacaaaacatccggaaccaccttaaatcaacgtccctcagaaagcctgtatgcgaagccacaatcctttccaacagaccatactaagt

SEQ ID NO: 55 - DNA, 20 bp, Artificial Sequence
  Sequence of diagnostic primer forward on ORFs of cassettes 120 and cassette 121
tctggcacactttcaccatc

SEQ ID NO: 56 - DNA, 20 bp, Artificial Sequence
  Sequence of diagnostic primer reverse on ORFs of cassettes 120 and cassette 121
tcaattcaccacgttcaggg

SEQ ID NO: 57 - DNA, 21 bp, Artificial Sequence
  Sequence of diagnostic primer forward on ORFs of cassette 137
gtaagaccaagaacccatggc

SEQ ID NO: 58 - DNA, 21 bp, Artificial Sequence
  Sequence of diagnostic primer forward on ORF of cassette 139
ttcttctgtcaagcacattcc

SEQ ID NO: 59 - DNA, 21 bp, Artificial Sequence
  Sequence of diagnostic primer reverse on terminator of cassette 137 and cassette 139
ccatggtaagtgacagtgcag

SEQ ID NO: 60 - DNA, 22 bp, Artificial Sequence
  Sequence of diagnostic primer forward on promoter of cassette 133 and cassette 135
gttgtctaacggatttctgcac

SEQ ID NO: 61 - DNA, 20 bp, Artificial Sequence
  Sequence of diagnostic primer forward on promoter of cassette 144 and cassette 146
cacttttccactcgcaacgg

SEQ ID NO: 62 - DNA, 24 bp, Artificial Sequence
  Sequence of diagnostic primer reverse on ORF of cassette 135 and cassette 146
cttcaacaattcaaccaaagcggc

SEQ ID NO: 63 - DNA, 21 bp, Artificial Sequence
  Sequence of diagnostic primer reverse on ORF of cassette 133 and cassette 144
gtatttagcagtggcagaagc